SEQUENCE LISTING (59 sequences)

SEQ ID NO: 1 - protein, 505 aa, Homo sapiens
MSYPGYPPPPGGYPPAAPGGGPWGGAAYPPPPSMPPIGLDNVATYAGQFNQDYLSGMAANMSGTFGGANMPNLYPGAPGAGYPPVPPGGFGQPPSAQQPVPPYGMYPPPGGNPPSRMPSYPPYPGAPVPGQPMPPPGQQPPGAYPGQPPVTYPGQPPVPLPGQQQPVPSYPGYPGSGTVTPAVPPTQFGSRGTITDAPGFDPLRDAEVLRKAMKGFGTDEQAIIDCLGSRSNKQRQQILLSFKTAYGKDLIKDLKSELSGNFEKTILALMKTPVLFDIYEIKEAIKGVGTDEACLIEILASRSNEHIRELNRAYKAEFKKTLEEAIRSDTSGHFQRLLISLSQGNRDESTNVDMSLAQRDAQELYAAGENRLGTDESKFNAVLCSRSRAHLVAVFNEYQRMTGRDIEKSICREMSGDLEEGMLAVVKCLKNTPAFFAERLNKAMRGAGTKDRTLIRIMVSRSETDLLDIRSEYKRMYGKSLYHDISGDTSGDYRKILLKICGGND

SEQ ID NO: 2 - protein, 323 aa, Homo sapiens
MASIWVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGAGTNEDALIEILTTRTSRQMKDISQAYYTVYKKSLGDDISSETSGDFRKALLTLADGRRDESLKVDEHLAKQDAQILYKAGENRWGTDEDKFTEILCLRSFPQLKLTFDEYRNISQKDIVDSIKGELSGHFEDLLLAIVNCVRNTPAFLAERLHRALKGIGTDEFTLNRIMVSRSEIDLLDIRTEFKKHYGYSLYSAIKSDTSGDYEITLLKICGGDD

SEQ ID NO: 3 - protein, 319 aa, Homo sapiens
MATKGGTVKAASGFNAMEDAQTLRKAMKGLGTDEDAIISVLAYRNTAQRQEIRTAYKSTIGRDLIDDLKSELSGNFEQVIVGMMTPTVLYDVQELRRAMKGAGTDEGCLIEILASRTPEEIRRISQTYQQQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAEKLYKSMKGLGTDDNTLIRVMVSRAEIDMLDIRAHFKRLYGKSLYSFIKGDTSGDYRKVLLVLCGGDD

SEQ ID NO: 4 - protein, 1299 aa, Homo sapiens
MSLQEMFRFPMGLLLGSVLLVASAPATLEPPGCSNKEQQVTVSHTYKIDVPKSALVQVDADPQPLSDDGASLLALGEAREEQNIIFRHNIRLQTPQKDCELAGSVQDLLARVKKLEEEMVEMKEQCSAQRCCQGVTDLSRHCSGHGTFSLETCSCHCEEGREGPACERLACPGACSGHGRCVDGRCLCHEPYVGADCGYPACPENCSGHGECVRGVCQCHEDFMSEDCSEKRCPGDCSGHGFCDTGECYCEEGFTGLDCAQVVTPQGLQLLKNTEDSLLVSWEPSSQVDHYLLSYYPLGKELSGKQIQVPKEQHSYEILGLLPGTKYIVTLRNVKNEVSSSPQHLLATTDLAVLGTAWVTDETENSLDVEWENPSTEVDYYKLRYGPMTGQEVAEVTVPKSSDPKSRYDITGLHPGTEYKITVVPMRGELEGKPILLNGRTEIDSPTNVVTDRVTEDTATVSWDPVQAVIDKYVVRYTSADGDTKEMAVHKDESSTVLTGLKPGEAYKVYVWAERGNQGSKKADTNALTEIDSPANLVTDRVTENTATISWDPVQATIDKYVVRYTSADDQETREVLVGKEQSSTVLTGLRPGVEYTVHVWAQKGDRESKKADTNAPTDIDSPKNLVTDRVTENMATVSWDPVQAAIDKYVVRYTSAGGETREVPVGKEQSSTVLTGLRPGMEYMVHVWAQKGDQESKKADTKAQTDIDSPQNLVTDRVTENMATVSWDPVRATIDRYVVRYTSAKDGETREVPVGKEQSSTVLTGLRPGVEYTVHVWAQKGAQESKKADTKAQTDIDSPQNLVTDWVTENTATVSWDPVQATIDRYVVHYTSANGETREVPVGKEQSSTVLTGLRPGMEYTVHVWAQKGNQESKKADTKAQTEIDGPKNLVTDWVTENMATVSWDPVQATIDKYMVRYTSADGETREVPVGKEHSSTVLTGLRPGMEYMVHVWAQKGAQESKKADTKAQTELDPPRNLRPSAVTQSGGILTWTPPSAQIHGYILTYQFPDGTVKEMQLGREDQRFALQGLEQGATYPVSLVAFKGGRRSRNVSTTLSTVGARFPHPSDCSQVQQNSNAASGLYTIYLHGDASRPLQVYCDMETDGGGWIVFQRRNTGQLDFFKRWRSYVEGFGDPMKEFWLGLDKLHNLTTGTPARYEVRVDLQTANESAYAIYDFFQVASSKERYKLTVGKYRGTAGDALTYHNGWKFTTFDRDNDIALSNCALTHHGGWWYKNCHLANPNGRYGETKHSEGVNWEPWKGHEFSIPYVELKIRPHGYSREPVLGRKKRTLRGRLRTF

SEQ ID NO: 5 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 6 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 7 - protein, 559 aa, Homo sapiens
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

SEQ ID NO: 8 - protein, 350 aa, Homo sapiens
MDSFNYTTPDYGHYDDKDTLDLNTPVDKTSNTLRVPDILALVIFAVVFLVGVLGNALVVWVTAFEAKRTINAIWFLNLAVADFLSCLALPILFTSIVQHHHWPFGGAACSILPSLILLNMYASILLLATISADRFLLVFKPIWCQNFRGAGLAWIACAVAWGLALLLTIPSFLYRVVREEYFPPKVLCGVDYSHDKRRERAVAIVRLVLGFLWPLLTLTICYTFILLRTWSRRATRSTKTLKVVVAVVASFFIFWLPYQVTGIMMSFLEPSSPTFLLLKKLDSLCVSFAYINCCINPIIYVVAGQGFQGRLRKSLPSLLRNVLTEESVVRESKSFTRSTVDTMAQKTQAV

SEQ ID NO: 9 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 10 - protein, 201 aa, Homo sapiens
MALSWVLTVLSLLPLLEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 11 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 12 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 13 - protein, 349 aa, Homo sapiens
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 14 - protein, 5762 aa, Homo sapiens
MGAPSACRTLVLALAAMLVVPQAETQGPVEPSWENAGHTMDGGAPTSSPTRRVSFVPPVTVFPSLSPLNPAHNGRVCSTWGDFHYKTFDGDVFRFPGLCNYVFSEHCRAAYEDFNVQLRRGLVGSRPVVTRVVIKAQGLVLEASNGSVLINGQREELPYSRTGLLVEQSGDYIKVSIRLVLTFLWNGEDSALLELDPKYANQTCGLCGDFNGLPAFNEFYAHNARLTPLQFGNLQKLDGPTEQCPDPLPLPAGNCTDEEGICHRTLLGPAFAECHALVDSTAYLAACAQDLCRCPTCPCATFVEYSRQCAHAGGQPRNWRCPELCPRTCPLNMQHQECGSPCTDTCSNPQRAQLCEDHCVDGCFCPPGTVLDDITHSGCLPLGQCPCTHGGRTYSPGTSFNTTCSSCTCSGGLWQCQDLPCPGTCSVQGGAHISTYDEKLYDLHGDCSYVLSKKCADSSFTVLAELRKCGLTDNENCLKAVTLSLDGGDTAIRVQADGGVFLNSIYTQLPLSAANITLFTPSSFFIVVQTGLGLQLLVQLVPLMQVFVRLDPAHQGQMCGLCGNFNQNQADDFTALSGVVEATGAAFANTWKAQAACANARNSFEDPCSLSVENENYARHWCSRLTDPNSAFSRCHSIINPKPFHSNCMFDTCNCERSEDCLCAALSSYVHACAAKGVQLSDWRDGVCTKYMQNCPKSQRYAYVVDACQPTCRGLSEADVTCSVSFVPVDGCTCPAGTFLNDAGACVPAQECPCYAHGTVLAPGEVVHDEGAVCSCTGGKLSCLGASLQKSTGCAAPMVYLDCSNSSAGTPGAECLRSCHTLDVGCFSTHCVSGCVCPPGLVSDGSGGCIAEEDCPCVHNEATYKPGETIRVDCNTCTCRNRRWECSHRLCLGTCVAYGDGHFITFDGDRYSFEGSCEYILAQDYCGDNTTHGTFRIVTENIPCGTTGTTCSKAIKLFVESYELILQEGTFKAVARGPGGDPPYKIRYMGIFLVIETHGMAVSWDRKTSVFIRLHQDYKGRVCGLCGNFDDNAINDFATRSRSVVGDALEFGNSWKLSPSCPDALAPKDPCTANPFRKSWAQKQCSILHGPTFAACRSQVDSTKYYEACVNDACACDSGGDCECFCTAVAAYAQACHDAGLCVSWRTPDTCPLFCDFYNPHGGCEWHYQPCGAPCLKTCRNPSGHCLVDLPGLEGCYPKCPPSQPFFNEDQMKCVAQCGCYDKDGNYYDVGARVPTAENCQSCNCTPSGIQCAHSLEACTCTYEDRTYSYQDVIYNTTDGLGACLIAICGSNGTIIRKAVACPGTPATTPFTFTTAWVPHSTTSPALPVSTVCVREVCRWSSWYNGHRPEPGLGGGDFETFENLRQRGYQVCPVLADIECRAAQLPDMPLEELGQQVDCDRMRGLMCANSQQSPPLCHDYELRVLCCEYVPCGPSPAPGTSPQPSLSASTEPAVPTPTQTTATEKTTLWVTPSIRSTAALTSQTGSSSGPVTVTPSAPGTTTCQPRCQWTEWFDEDYPKSEQLGGDVESYDKIRAAGGHLCQQPKDIECQAESFPNWTLAQVGQKVHCDVHFGLVCRNWEQEGVFKMCYNYRIRVLCCSDDHCRGRATTPPPTTELETATTTTTQALFSTPQPTSSPGLTRAPPASTTAVPTLSEGLTSPRYTSTLGTATTGGPTTPAGSTEPTVPGVATSTLPTRSALPGTTGSLGTWRPSQPPTLAPTTMATSRARPTGTASTASKEPLTTSLAPTLTSELSTSQAETSTPRTETTMSPLTNTTTSQGTTRCQPKCEWTEWFDVDFPTSGVAGGDMETFENIRAAGGKMCWAPKSIECRAENYPEVSIDQVGQVLTCSLETGLTCKNEDQTGRFNMCFNYNVRVLCCDDYSHCPSTPATSSTATPSSTPGTTWILTKPTTTATTTASTGSTATPTSTLRTAPPPKVLTTTATTPTVTSSKATPSSSPGTATALPALRSTATTPTATSVTPIPSSSLGTTWTRLSQTTTPTATMSTATPSSTPETAHTSTVLTATATTTGATGSVATPSSTPGTAHTTKVPTTTTTGFTATPSSSPGTALTPPVWISTTTTPTTRGSTVTPSSIPGTTHTATVLTTTTTTVATGSMATPSSSTQTSGTPPSLTTTATTITATGSTTNPSSTPGTTPIPPVLTTTATTPAATSNTVTPSSALGTTHTPPVPNTMATTHGRSLPPSSPHTVRTAWTSATSGILGTTHITEPSTVTSHTLAATTGTTQHSTPALSSPHPSSRTTESPPSPGTTTPGHTTATSRTTATATPSKTRTSTLLPSSPTSAPITTVVTMGCEPQCAWSEWLDYSYPMPGPSGGDFDTYSNIRAAGGAVCEQPLGLECRAQAQPGVPLRELGQVVECSLDFGLVCRNREQVGKFKMCFNYEIRVFCCNYGHCPSTPATSSTAMPSSTPGTTWILTELTTTATTTESTGSTATPSSTPGTTWILTEPSTTATVTVPTGSTATASSTQATAGTPHVSTTATTPTVTSSKATPFSSPGTATALPALRSTATTPTATSFTAIPSSSLGTTWTRLSQTTTPTATMSTATPSSTPETVHTSTVLTTTATTTGATGSVATPSSTPGTAHTTKVLTTTTTGFTATPSSSPGTARTLPVWISTTTTPTTRGSTVTPSSIPGTTHTPTVLTTTTTTVATGSMATPSSSTQTSGTPPSLTTTATTITATGSTTNPSSTPGTTPIPPVLTTTATTPAATSSTVTPSSALGTTHTPPVPNTTATTHGRSLSPSSPHTVRTAWTSATSGTLGTTHITEPSTGTSHTPAATTGTTQHSTPALSSPHPSSRTTESPPSPGTTTPGHTRATSRTTATATPSKTRTSTLLPSSPTSAPITTVVTMGCEPQCAWSEWLDYSYPMPGPSGGDFDTYSNIRAAGGAVCEQPLGLECRAQAQPGVPLRELGQVVECSLDFGLVCRNREQVGKFKMCFNYEIRVFCCNYGHCPSTPATSSTATPSSTPGTTWILTEQTTAATTTATTGSTAIPSSTPGTAPPPKVLTSTATTPTATSSKATSSSSPRTATTLPVLTSTATKSTATSFTPIPSFTLGTTGTLPEQTTTPMATMSTIHPSSTPETTHTSTVLTTKATTTRATSSMSTPSSTPGTTWILTELTTAATTTAATGPTATPSSTPGTTWILTEPSTTATVTVPTGSTATASSTRATAGTLKVLTSTATTPTVISSRATPSSSPGTATALPALRSTATTPTATSVTAIPSSSLGTAWTRLSQTTTPTATMSTATPSSTPETVHTSTVLTTTTTTTRATGSVATPSSTPGTAHTTKVPTTTTTGFTATPSSSPGTALTPPVWISTTTTPTTRGSTVTPSSIPGTTHTATVLTTTTTTVATGSMATPSSSTQTSGTPPSLTTTATTITATGSTTNPSSTPGTTPIPPVLTTTATTPAATSSTVTPSSALGTTHTPPVPNTTATTHGRSLPPSSPHTVRTAWTSATSGILGTTHITEPSTVTSHTPAATTSTTQHSTPALSSPHPSSRTTESPPSPGTTTPGHTRGTSRTTATATPSKTRTSTLLPSSPTSAPITTVVTTGCEPQCAWSEWLDYSYPMPGPSGGDFDTYSNIRAAGGAVCEQPLGLECRAQAQPGVPLRELGQVVECSLDFGLVCRNREQVGKFKMCFNYEIRVFCCNYGHCPSTPATSSTATPSSTPGTTWILTKLTTTATTTESTGSTATPSSTPGTTWILTEPSTTATVTVPTGSTATASSTQATAGTPHVSTTATTPTVTSSKATPFSSPGTATALPALRSTATTPTATSFTAIPSSSLGTTWTRLSQTTTPTATMSTATPSSTPETAHTSTVLTTTATTTRATGSVATPSSTPGTAHTTKVPTTTTTGFTVTPSSSPGTARTPPVWISTTTTPTTSGSTVTPSSVPGTTHTPTVLTTTTTTVATGSMATPSSSTQTSGTPPSLITTATTITATGSTTNPSSTPGTTPIPPVLTTTATTPAATSSTVTPSSALGTTHTPPVPNTTATTHGRSLSPSSPHTVRTAWTSATSGTLGTTHITEPSTGTSHTPAATTGTTQHSTPALSSPHPSSRTTESPPSPGTTTPGHTTATSRTTATATPSKTRTSTLLPSSPTSAPITTVVTTGCEPQCAWSEWLDYSYPMPGPSGGDFDTYSNIRAAGGAVCEQPLGLECRAQAQPGVPLGELGQVVECSLDFGLVCRNREQVGKFKMCFNYEIRVFCCNYGHCPSTPATSSTAMPSSTPGTTWILTELTTTATTTASTGSTATPSSTPGTAPPPKVLTSPATTPTATSSKATSSSSPRTATTLPVLTSTATKSTATSVTPIPSSTLGTTGTLPEQTTTPVATMSTIHPSSTPETTHTSTVLTTKATTTRATSSTSTPSSTPGTTWILTELTTAATTTAATGPTATPSSTPGTTWILTELTTTATTTASTGSTATPSSTPGTTWILTEPSTTATVTVPTGSTATASSTQATAGTPHVSTTATTPTVTSSKATPSSSPGTATALPALRSTATTPTATSFTAIPSSSLGTTWTRLSQTTTPTATMSTATPSSTPETVHTSTVLTATATTTGATGSVATPSSTPGTAHTTKVPTTTTTGFTATPSSSPGTALTPPVWISTTTTPTTTTPTTSGSTVTPSSIPGTTHTARVLTTTTTTVATGSMATPSSSTQTSGTPPSLTTTATTITATGSTTNPSSTPGTTPITPVLTSTATTPAATSSKATSSSSPRTATTLPVLTSTATKSTATSFTPIPSSTLWTTWTVPAQTTTPMSTMSTIHTSSTPETTHTSTVLTTTATMTRATNSTATPSSTLGTTRILTELTTTATTTAATGSTATLSSTPGTTWILTEPSTIATVMVPTGSTATASSTLGTAHTPKVVTTMATMPTATASTVPSSSTVGTTRTPAVLPSSLPTFSVSTVSSSVLTTLRPTGFPSSHFSTPCFCRAFGQFFSPGEVIYNKTDRAGCHFYAVCNQHCDIDRFQGACPTSPPPVSSAPLSSPSPAPGCDNAIPLRQVNETWTLENCTVARCVGDNRVVLLDPKPVANVTCVNKHLPIKVSDPSQPCDFHYECECICSMWGGSHYSTFDGTSYTFRGNCTYVLMREIHARFGNLSLYLDNHYCTASATAAAARCPRALSIHYKSMDIVLTVTMVHGKEEGLILFDQIPVSSGFSKNGVLVSVLGTTTMRVDIPALGVSVTFNGQVFQARLPYSLFHNNTEGQCGTCTNNQRDDCLQRDGTTAASCKDMAKTWLVPDSRKDGCWAPTGTPPTASPAAPVSSTPTPTPCPPQPLCDLMLSQVFAECHNLVPPGPFFNACISDHCRGRLEVPCQSLEAYAELCRARGVCSDWRGATGGLCDLTCPPTKVYKPCGPIQPATCNSRNQSPQLEGMAEGCFCPEDQILFNAHMGICVQACPCVGPDGFPKFPGERWVSNCQSCVCDEGSVSVQCKPLPCDAQGQPPPCNRPGFVTVTRPRAENPCCPETVCVCNTTTCPQSLPVCPPGQESICTQEEGDCCPTFRCRPQLCSYNGTFYGVGATFPGALPCHMCTCLSGDTQDPTVQCQEDACNNTTCPQGFEYKRVAGQCCGECVQTACLTPDGQPVQLNETWVNSHVDNCTVYLCEAEGGVHLLTPQPASCPDVSSCRGSLRKTGCCYSCEEDSCQVRINTTILWHQGCETEVNITFCEGSCPGASKYSAEAQAMQHQCTCCQERRVHEETVPLHCPNGSAILHTYTHVDECGCTPFCVPAPMAPPHTRGFPAQEATAV

SEQ ID NO: 15 - protein, 217 aa, Homo sapiens [sequence omitted]

SEQ ID NO: 16 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 17 - protein, 510 aa, Homo sapiens
MRPGLSFLLALLFFLGQAAGDLGDVGPPIPSPGFSSFPGVDSSSSFSSSSRSGSSSSRSLGSGGSVSQLFSNFTGSVDDRGTCQCSVSLPDTTFPVDRVERLEFTAHVLSQKFEKELSKVREYVQLISVYEKKLLNLTVRIDIMEKDTISYTELDFELIKVEVKEMEKLVIQLKESFGGSSEIVDQLEVEIRNMTLLVEKLETLDKNNVLAIRREIVALKTKLKECEASKDQNTPVVHPPPTPGSCGHGGVVNISKPSVVQLNWRGFSYLYGAWGRDYSPQHPNKGLYWVAPLNTDGRLLEYYRLYNTLDDLLLYINARELRITYGQGSGTAVYNNNMYVNMYNTGNIARVNLTTNTIAVTQTLPNAAYNNRFSYANVAWQDIDFAVDENGLWVIYSTEASTGNMVISKLNDTTLQVLNTWYTKQYKPSASNAFMVCGVLYATRTMNTRTEEIFYYYDTNTGKEGKLDIVMHKMQEKVQSINYNPFDQKLYVYNDGYLLNYDLSVLQKPQ

SEQ ID NO: 18 - protein, 541 aa, Homo sapiens
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVM

SEQ ID NO: 19 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 20 - protein, 639 aa, Homo sapiens
MSARGPAIGIDLGTTYSCVGVFQHGKVEIIANDQGNRTTPSYVAFTDTERLIGDAAKNQVAMNPTNTIFDAKRLIGRKFEDATVQSDMKHWPFRVVSEGGKPKVQVEYKGETKTFFPEEISSMVLTKMKEIAEAYLGGKVHSAVITVPAYFNDSQRQATKDAGTITGLNVLRIINEPTAAAIAYGLDKKGCAGGEKNVLIFDLGGGTFDVSILTIEDGIFEVKSTAGDTHLGGEDFDNRMVSHLAEEFKRKHKKDIGPNKRAVRRLRTACERAKRTLSSSTQASIEIDSLYEGVDFYTSITRARFEELNADLFRGTLEPVEKALRDAKLDKGQIQEIVLVGGSTRIPKIQKLLQDFFNGKELNKSINPDEAVAYGAAVQAAILIGDKSENVQDLLLLDVTPLSLGIETAGGVMTPLIKRNTTIPTKQTQTFTTYSDNQSSVLVQVYEGERAMTKDNNLLGKFDLTGIPPAPRGVPQIEVTFDIDANGILNVTAADKSTGKENKITITNDKGRLSKDDIDRMVQEAERYKSEDEANRDRVAAKNALESYTYNIKQTVEDEKLRGKISEQDKNKILDKCQEVINWLDRNQMAEKDEYEHKQKELERVCNPIISKLYQGGPGGGSGGGGSGASGGPTIEEVD

SEQ ID NO: 21 - protein, 241 aa, Homo sapiens
MFLTRSEYDRGVNTFSPEGRLFQVEYAIEAIKLGSTAIGIQTSEGVCLAVEKRITSPLMEPSSIEKIVEIDAHIGCAMSGLIADAKTLIDKARVETQNHWFTYNETMTVESVTQAVSNLALQFGEEDADPGAMSRPFGVALLFGGVDEKGPQLFHMDPSGTFVQCDARAIGSASEGAQSSLQEVYHKSMTLKEAIKSSLIILKQVMEEKLNATNIELATVQPGQNFHMFTKEELEEVIKDI

SEQ ID NO: 22 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 23 - protein, 13 aa, Artificial
  Synthetic
GTITDAPGFDPLR

SEQ ID NO: 24 - protein, 9 aa, Artificial
  Synthetic
SETDLLDIR

SEQ ID NO: 25 - protein, 16 aa, Artificial
  Synthetic
GAGTNEDALIEILTTR

SEQ ID NO: 26 - protein, 13 aa, Artificial
  Synthetic
SDTSGDYEITLLK

SEQ ID NO: 27 - protein, 16 aa, Artificial
  Synthetic
GAGTDEGCLIEILASR

SEQ ID NO: 28 - protein, 12 aa, Artificial
  Synthetic
DEGNYLDDALVR

SEQ ID NO: 29 - protein, 8 aa, Artificial
  Synthetic
EEQNIIFR

SEQ ID NO: 30 - protein, 9 aa, Artificial
  Synthetic
AQTEIDGPK

SEQ ID NO: 31 - protein, 10 aa, Artificial
  Synthetic
LLNENSYVPR

SEQ ID NO: 32 - protein, 13 aa, Artificial
  Synthetic
VSASPLLYTLIEK

SEQ ID NO: 33 - protein, 8 aa, Artificial
  Synthetic
VTILELFR

SEQ ID NO: 34 - protein, 10 aa, Artificial
  Synthetic
TFDEIASGFR

SEQ ID NO: 35 - protein, 11 aa, Artificial
  Synthetic
LSPIYNLVPVK

SEQ ID NO: 36 - protein, 12 aa, Artificial
  Synthetic
AIEDYINEFSVR

SEQ ID NO: 37 - protein, 7 aa, Artificial
  Synthetic
SLPSLLR

SEQ ID NO: 38 - protein, 10 aa, Artificial
  Synthetic
NVLTEESVVR

SEQ ID NO: 39 - protein, 8 aa, Artificial
  Synthetic
VLEDSDLK

SEQ ID NO: 40 - protein, 14 aa, Artificial
  Synthetic
SDIDEIVLVGGSTR

SEQ ID NO: 41 - protein, 15 aa, Artificial
  Synthetic
YVGGQEHFAHLLILR

SEQ ID NO: 42 - protein, 14 aa, Artificial
  Synthetic
EQLGEFYEALDCLR

SEQ ID NO: 43 - protein, 12 aa, Artificial
  Synthetic
QSTLVLFPGDLR

SEQ ID NO: 44 - protein, 9 aa, Artificial
  Synthetic
FQTFEGDLK

SEQ ID NO: 45 - protein, 14 aa, Artificial
  Synthetic
LEIQLLENSLSTYK

SEQ ID NO: 46 - protein, 9 aa, Artificial
  Synthetic
ENLQGLVTR

SEQ ID NO: 47 - protein, 14 aa, Artificial
  Synthetic
EVLLLVHNLPQDPR

SEQ ID NO: 48 - protein, 9 aa, Artificial
  Synthetic
LFIPNITTK

SEQ ID NO: 49 - protein, 16 aa, Artificial
  Synthetic
AAGGAVCEQPLGLECR

SEQ ID NO: 50 - protein, 19 aa, Artificial
  Synthetic
VCGLCGNFDDNAINDFATR

SEQ ID NO: 51 - protein, 7 aa, Artificial
  Synthetic
YFLWVVK

SEQ ID NO: 52 - protein, 7 aa, Artificial
  Synthetic
NQQIFLR

SEQ ID NO: 53 - protein, 9 aa, Artificial
  Synthetic
SEIDLFNIR

SEQ ID NO: 54 - protein, 12 aa, Artificial
  Synthetic
VQSINYNPFDQK

SEQ ID NO: 55 - protein, 12 aa, Artificial
  Synthetic
GPAGDATVASEK

SEQ ID NO: 56 - protein, 14 aa, Artificial
  Synthetic
LFGGNFAHQASVAR

SEQ ID NO: 57 - protein, 11 aa, Artificial
  Synthetic
NALESYTYNIK

SEQ ID NO: 58 - protein, 14 aa, Artificial
  Synthetic
PFGVALLFGGVDEK

SEQ ID NO: 59 - protein, 7 aa, Artificial
  Synthetic
ELTSELK

(19) United States
(12) Patent Application Publication
(10) Pub. No.: US 2020/0124604 A1
(43) Pub. Date: Apr. 23, 2020

(54) BIOMARKER FOR DETECTING COLORECTAL CANCER

(71) Applicants: National Institutes of Biomedical Innovation, Health and Nutrition, Ibaraki-shi, Osaka (JP); DENKA SEIKEN CO., LTD., Chuo-ku, Tokyo (JP)

(72) Inventors: Takeshi Tomonaga, Osaka (JP); Takashi SHIROMIZU, Osaka (JP)

(73) Assignees: National Institutes of Biomedical Innovation, Health and Nutrition, Ibaraki-shi, Osaka (JP); DENKA SEIKEN CO., LTD., Chuo-ku, Tokyo (JP)

(21) Appl. No.: 16/625,697

(22) PCT Filed: Jun. 29, 2018
(86) PCT No.: PCT/JP2018/024806

§ 371 Date: Dec. 21, 2019

(30) Foreign Application Priority Data
Jun. 30, 2017 (JP) 2017-129941

(51) Int. Cl. G01N 33/574; G01N 33/68

(52) U.S. Cl. CPC G01N 33/57419; G01N 33/6848

(57) ABSTRACT

Provided is a biomarker for detecting colorectal cancer in an early stage. A colorectal cancer biomarker for detecting colorectal cancer, wherein the biomarker consists of at least one protein of the following 22 proteins with numbers 1 to 22, or at least one peptide of the partial peptides of the proteins with numbers 1 to 22: 1. Annexin A11; 2. Annexin A3; 3. Annexin A4; 4. Tanascin-N; 5. Transferrin receptor protein 1; 6. Glucose transporter 1; 7. Complement component C9; 8. CD88 antigen; 9. 78 kDa glucose-regulated protein; 10. α-1-acid glycoprotein; 11. Matrix metalloproteinase-9; 12. Angiopoietin-1; 13. CD67 antigen; 14. Mucin-5B; 15. Adapter protein GRB2; 16. Annexin A5; 17. Olfactomedin-4; 18. Neutral amino acid transporter B(0); 19. Tripeptidyl-peptidase 1; 20. Heat shock-related 70 kDa protein 2; 21. Proteasome subunit α type-5; or 22. Neutrophil gelatinase-associated lipocalin.

### TECHNICAL FIELD

The present invention relates to a novel protein or peptide biomarker that can be used in detection of colorectal cancer.

### BACKGROUND ART

Colorectal cancer (CRC) is one of the most common cancers in the world, and thus, the development of a biomarker effective for CRC is essential for the improvement of human survival rate. However, the currently used methods for diagnosing colorectal cancer have involved biomarkers with extremely low accuracy, such as faecal occult blood or CEA, and novel biomarkers capable of diagnosing colorectal cancer in an early stage with high accuracy have not been discovered. Hence, it has been desired to develop a novel blood biomarker.

As a result of large-scale omics-based studies, a large number of cancer-related factors and cancer biomarker candidates have been discovered. Nevertheless, clinically applied blood biomarkers have not yet been present. As evidence to support this fact, the number of biomarkers recently approved by United States Food and Drug Administration (FDA) is extremely low (2 biomarkers or less every year). One of the causes of this stagnation is considered to be a serious problem in the strategy of biomarker development.

Conventionally, in many clinical tests, detection of a protein biomarker has been carried out according to antibody-based quantitative analyses (in which the enzyme-linked immunosorbent assay (ELISA) has been mainly applied). These methods largely depend on the quality of an antibody, and thus, the methods are problematic in terms of accuracy, in particular, specificity. Moreover, according to these methods, it is difficult to simultaneously evaluate marker candidates for multiple items.

As a result of recent progression of mass spectrometers (MS), it has become possible that clinical test methods will largely change. As a reason therefor, a selected reaction monitoring/multiple reaction monitoring method (SRM/MRM), which is a representative target proteomics method, cannot only quantify a biomarker protein with high accuracy, but also, can simultaneously quantify markers for multiple items. According to the previous report, Whiteaker et al. had quantified breast cancer biomarker candidate proteins for multiple items present in the plasma of a patient according to the SRM/MRM method (see Non Patent Literature 1). Other than this, several cases of examining various cancer biomarker candidate proteins according to the SRM/MRM method, including the report by the present inventors' group, have been reported (see Non Patent Literatures 2 to 4).

Thus, the target proteomics using the SRM/MRM method could be a strong tool for discovering biomarkers.

### CITATION LIST

### Non Patent Literature

Non Patent Literature 1: Whiteaker, J. et al., Nat. Blotechnol. 29, 625-634 (2011)
Non Patent Literature 2: Kume, H. et al., Mol. Cell. Proteomics 13, 1471-1484 (2014)
Non Patent Literature 3: Muraoka, S. et al., J. Proteome Res. 11, 4201-4210 (2012)
Non Patent Literature 4: Narumi, R. et al., J. Proteome Res. 11, 5311-5322 (2012)

### SUMMARY OF INVENTION

### Technical Problem

It is an object of the present invention to provide a biomarker for detecting colorectal cancer in an early stage.

### Solution to Problem

The present inventors have first searched for CRC biomarker candidate proteins by the document retrieval of PubMed. In order to detect such biomarker candidate proteins in blood, the inventors have focused on extracellular vesicles (EV) present in blood. This is because EV has recently attracted attention as a mediator for intercellular communication, and also because EV is said to be associated with the onset or progression of various diseases and is considered to comprise many biomarker candidates. When a trace amount protein is to be detected in blood using a mass spectrometer, detection of such a trace amount of marker candidate protein may be inhibited by the presence of a large amount of contaminant protein in blood, such as albumin, in some cases. However, since such a large amount of contaminant protein is removed when EV is purified from the blood, it becomes possible to detect a trace amount of biomarker candidate protein in the EV. Accordingly, such a protein contained in EV has been considered to be a promising biomarker candidate.

The present inventors have narrowed down the biomarker candidate proteins discovered by the document retrieval to proteins likely to be present in blood EV, and have then quantified those proteins according to the SRM/MRM method. As a result, the inventors have specified several biomarker candidates for use in the early-stage diagnosis of CRC, thereby completing the present invention.

Specifically, the aspects of the present invention are as follows.

[1] A colorectal cancer biomarker for detecting colorectal cancer, wherein the biomarker consists of at least one protein of the following 22 proteins with numbers 1 to 22, or at least one peptide of the partial peptides of the proteins with numbers 1 to 22:

### 1. Annexin A11 (ANXA 11) (SEQ ID NO: 1);

### 2. Annexin A3 (ANXA 3) (SEQ ID NO: 2);

### 3. Annexin A4 (ANXA 4) (SEQ ID NO: 3);

### 4. Tanascin-N(TNN) (SEQ ID NO: 4);

5. Transferrin receptor protein 1 (TFRC) (SEQ ID NO: 5);
6. Glucose transporter 1(GLUT-1) (SLC2A1) (SEQ ID NO: 6);
7. Complement component C9 (C9) (SEQ ID NO: 7);
8. CD88 antigen (C5AR1) (SEQ ID NO: 8);
9. 78 kDa glucose-regulated protein (HSPA5) (SEQ ID NO: 9);
10. α-1-acid glycoprotein (ORM1) (SEQ ID NO: 10);
11. Matrix metalloproteinase-9 (MMP9) (SEQ ID NO: 11);

### 12. Angiopoietin-1 (ANGPT1) (SEQ ID NO: 12);

13. CD67 antigen (CEACAM8) (SEQ ID NO: 13);

### 14. Mucin-5B (MUC5B) (SEQ ID NO: 14);

15. Adapter protein GRB2 (GRB2) (SEQ ID NO: 15);

### 16. Annexin A5(Annexin A5)(ANXA 5) (SEQ ID NO: 16);

### 17. Olfactomedin-4 (OLFM4) (SEQ ID NO: 17);

18. Neutral amino acid transporter B(0) (SLC1A5) (SEQ ID NO: 18);

### 19. Tripeptidyl-peptidase 1 (TPP1) (SEQ ID NO: 19);

20. Heat shock-related 70 kDa protein 2 (HSPA2) (SEQ ID NO: 20);
21. Proteasome subunit α type-5 (PSMA5) (SEQ ID NO: 21); or
22. Neutrophil gelatinase-associated lipocalin (LCN2) (SEQ ID NO: 22).
[2] The colorectal cancer biomarker for detecting colorectal cancer according to the above [1], which consists of a combination of two or more proteins of the 22 proteins with numbers 1 to 22 according to the above [1], or two or more peptides of the partial peptides of the proteins with numbers 1 to 22.
[3] The colorectal cancer biomarker for detecting colorectal cancer according to the above [1] or [2], wherein the partial peptides of the proteins with numbers 1 to 22 according to the above [1] are peptides consisting of the amino acid sequences as set forth in SEQ ID NOS: 23 to 59.
[4] The colorectal cancer biomarker for detecting colorectal cancer according to the above [1], which consists of at least one protein of the following 12 proteins, or at least one peptide of the partial peptides of the 12 proteins:

### 1. Annexin A11 (SEQ ID NO: 1);

### 2. Annexin A3 (SEQ ID NO: 2);

### 3. Annexin A4 (SEQ ID NO: 3);

### 4. Tanascin-N(SEQ ID NO: 4);

5. Transferrin receptor protein 1 (SEQ ID NO: 5);
6. Glucose transporter 1 (SEQ ID NO: 6);
8. CD88 antigen (SEQ ID NO: 8);
11. Matrix metalloproteinase-9 (SEQ ID NO: 11);

### 16. Annexin A5 (SEQ ID NO: 16);

### 17. Olfactomedin-4 (SEQ ID NO: 17);

### 19. Tripeptidyl-peptidase 1 (SEQ ID NO: 19); or

22. Neutrophil gelatinase-associated lipocalin (SEQ ID NO: 22).
[5] The colorectal cancer biomarker for detecting colorectal cancer according to the above [4], which consists of a combination of two or more proteins of the 12 proteins according to the above [4], or two or more peptides of the partial peptides of the 12 proteins.
[6] The colorectal cancer biomarker for detecting colorectal cancer according to the above [4] or [5], wherein the partial peptides of the 12 proteins according to the above [4] are peptides consisting of the amino acid sequences as set forth in SEQ ID NOS: 23 to 34, 37, 38, 43, 44, 53, 54, 56 and 59.
[7] The colorectal cancer biomarker for detecting colorectal cancer according to the above [1], which consists of Annexin A4 or Annexin A11, or a partial peptide thereof.
[8] The colorectal cancer biomarker for detecting colorectal cancer according to the above [7], which consists of a combination of Annexin A4 and Annexin A11, or partial peptides of Annexin A4 and partial peptides of Annexin A11.
[9] The colorectal cancer biomarker for detecting colorectal cancer according to the above [7] or [8], wherein the partial peptides of Annexin A4 and Annexin A11 are peptides consisting of the amino acid sequences as set forth in SEQ ID NOS: 23, 24, 27 and 28.
[10] A colorectal cancer biomarker for detecting colorectal cancer, which consists of a combination of the colorectal cancer biomarker according to any one of the above [1] to [9] and CEA.
[11] A method of detecting colorectal cancer, comprising measuring the colorectal cancer biomarker according to any one of the above [1] to [10] in a biological sample.
[12] A method of detecting colorectal cancer, comprising measuring the colorectal cancer biomarker according to any one of the above [1] to [10] in a biological sample, and then determining that the subject is affected with colorectal cancer when the biomarker is present in a higher concentration in the biological sample than in a healthy subject.
[13] The method of detecting colorectal cancer according to the above [11] or [12], wherein the biological sample is an extracellular vesicle (EV) in blood.
[14] The method of detecting colorectal cancer according to any one of the above [11] to [13], wherein the detection is carried out by an immunoassay or a mass spectrometry.

A kit of detecting colorectal cancer, comprising an antibody reacting against the colorectal cancer biomarker according to any one of the above [1] to [10] in the biological sample.

The present application claims priority from Japanese Patent Application No. 2017-129941; the disclosure of which is hereby incorporated by reference.

### Advantageous Effects of Invention

According to the biomarker of the present invention, colorectal cancer can be detected with high sensitivity and high specificity, and in particular, early-stage colorectal cancer can be detected. Furthermore, by combining the present biomarkers with one another, colorectal cancer can be detected with higher sensitivity and higher specificity.

### BRIEF DESCRIPTION OF DRAWINGS

FIG. 1 is a view showing a strategy for searching for a colorectal cancer marker in an extracellular vesicle (EV). In the figure, the number of candidate proteins selected in each step is shown.

FIG. 2 is a view showing a method of shotgun proteomics analysis for CRC biomarker candidate proteins in EV. FIG. 2a shows a step of preparation of EV and MS analysis, and FIG. 2b shows the results of Venn diagram analysis performed on biomarker candidate proteins and EV proteins, which had been identified by the shotgun proteome analysis. Among the EV proteins identified from serum or cell culture supernatant, 356 colorectal cancer biomarker candidate proteins were identified to be EV proteins.

FIG. 3 is a table showing SRM target proteins (46) and peptides (71).

FIG. 4 is a table showing a list of proteins verified with high accuracy (AUC>0.7).

FIG. 5-1 is a view showing the results of relative quantification performed on serum peptides among 3 groups (N, C and Cm) according to SRM analysis. N indicates non-cancerous control patients, C indicates non-metastatic cancer patients, and Cm indicates metastatic cancer patients. The dot plot graph shows the peak area ratio of endogenous peptides to SI peptides (*; p<0.05, **; p<0.01, N.S; no significant difference). The longitudinal axis indicates Area Ratio.

FIG. 5-2 is a view showing the results of relative quantification performed on peptides among 3 groups (N, C and Cm) according to SRM analysis (continuation of FIG. 5-1).

FIG. 5-3 is a view showing the results of relative quantification performed on peptides among 3 groups (N, C and Cm) according to SRM analysis (continuation of FIG. 5-2).

FIG. 5-4 is a view showing the results of relative quantification performed on peptides among 3 groups (N, C and Cm) according to SRM analysis (continuation of FIG. 5-3).

FIG. 5-5 is a view showing the results of relative quantification performed on peptides among 3 groups (N, C and Cm) according to SRM analysis (continuation of FIG. 5-4).

FIG. 5-6 is a view showing the results of relative quantification performed on peptides among 3 groups (N, C and Cm) according to SRM analysis (continuation of FIG. 5-5).

FIG. 6-1 is a view showing the results of the statistical analysis of target peptides (Part 1). FIG. 6-1a shows the results of relative quantification performed on peptides among 3 groups (N, C and Cm) according to SRM analysis. N indicates non-cancerous control, C indicates non-metastatic cancer, and Cm indicates metastatic cancer. The dot plot graph shows the peak area ratio of endogenous peptides to SI peptides (*; p<0.05, **; p<0.01, N.S; no significant difference). The longitudinal axis indicates Area Ratio. FIG. 6-1b shows the results (1) of ROC curve analysis for discriminating between N and C, and the results (2) of ROC curve analysis for discriminating between C and Cm. The area under the curve (AUC) for discrimination is shown in each graph. The longitudinal axis indicates sensitivity, and the horizontal axis indicates 1-specificity.

FIG. 6-2 is a view showing the results of the statistical analysis of target peptides (Part 2). FIG. 6-2c shows the results of relative quantification performed on peptides among 3 groups (N, C and Cm) according to SRM analysis. N indicates non-cancerous control, C indicates non-metastatic cancer, and Cm indicates metastatic cancer. The dot plot graph shows the peak area ratio of endogenous peptides to SI peptides (*; p<0.05, **; p<0.01, N.S; no significant difference). The longitudinal axis indicates Area Ratio. FIG. 6-2d shows the results (1) of ROC curve analysis for discriminating between N and C and the results (2) of ROC curve analysis for discriminating between C and Cm. The area under the curve (AUC) for discrimination is shown in each graph. The longitudinal axis indicates sensitivity, and the horizontal axis indicates 1-specificity.

FIG. 7-1 is a view showing ROC curve (1) for discriminating between N and C, and ROC curve (2) for discriminating between C and Cm (Part 1). The area under the curve (AUC) for discrimination is shown in each graph. The longitudinal axis indicates sensitivity, and the horizontal axis indicates 1-specificity.

FIG. 7-2 is a view showing ROC curve (1) for discriminating between N and C, and ROC curve (2) for discriminating between C and Cm (continuation of FIG. 7-1).

FIG. 7-3 is a view showing ROC curve (1) for discriminating between N and C, and ROC curve (2) for discriminating between C and Cm (continuation of FIG. 7-2).

FIG. 7-4 is a view showing ROC curve (1) for discriminating between N and C, and ROC curve (2) for discriminating between C and Cm (continuation of FIG. 7-3).

FIG. 7-5 is a view showing ROC curve (1) for discriminating between N and C, and ROC curve (2) for discriminating between C and Cm (continuation of FIG. 7-4).

FIG. 7-6 is a view showing ROC curve (1) for discriminating between N and C, and ROC curve (2) for discriminating between C and Cm (continuation of FIG. 7-5).

FIG. 8-1 is a view showing the results of ROC curve analysis performed on the combinations of target peptides (Part 1). The diagnostic sensitivity obtained by the combination of peptides was evaluated between N and C. The area under the curve (AUC), sensitivity and specificity are shown in each graph.

FIG. 8-2 is a view showing the results of ROC curve analysis performed on the combinations of target peptides (Part 2). The diagnostic sensitivity obtained by the combination of peptides was evaluated between N and C. The area under the curve (AUC), sensitivity and specificity are shown in each graph.

FIG. 9 is a view showing the results of ROC curve analysis performed on the combinations of target peptides. The diagnostic sensitivity obtained by the combination of peptides was evaluated between N and C. The area under the curve (AUC), sensitivity and specificity are shown in each graph. FIG. 9 shows combinations having high accuracy (AUC>0.9). Other combinations are shown in FIG. 8-1 and FIG. 8-2.

FIG. 10-1 is a view showing the results of a comparison between the target peptides and CEA in terms of sensitivity. When the cutoff value is set to be in the maximum peak area of N, the sensitivity is calculated to be the ratio of the sample (wherein the dotted line indicates a specificity of 100%). The longitudinal axis indicates Area Ratio.

FIG. 10-2 is a view showing the results of a comparison between the target peptides and CEA in terms of sensitivity (continuation of FIG. 10-1).

FIG. 10-3 is a view showing the results of a comparison between the target peptides and CEA in terms of sensitivity (continuation of FIG. 10-2).

FIG. 11 is a view showing the results of a comparison between the target peptides and CEA in terms of sensitivity. When the cutoff value (Cutoff) is set to be in the maximum peak area of N, the sensitivity is calculated to be the ratio of the sample (wherein the dotted line indicates a specificity of 100%). FIG. 11 shows top three peptides having high sensitivity in discrimination between N and C. The longitudinal axis of the graphs regarding the three peptides indicates Area Ratio. Other peptides having higher sensitivity than CEA are shown in FIGS. 10-1 to 10-3.

### DESCRIPTION OF EMBODIMENTS

Hereinafter, the present invention will be described in detail.

The present invention relates to a protein used as a biomarker for detecting colorectal cancer (CRC), or a partial peptide thereof. In addition, the present invention relates to a method of detecting colorectal cancer, using the biomarker. Moreover, the present invention relates to a method for obtaining auxiliary data for use in diagnosing colorectal cancer using the biomarker. By using the biomarker of the present invention, it is possible to detect colorectal cancer in an early stage.

The biomarker used in the present invention can be searched, for example, according to proteome analysis technology. For example, the biomarker used in the present can be searched by the following process.

First, using the tissues of colorectal cancer patients, proteins that become biomarker candidates are searched according to quantitative shotgun proteomics. In this searching, comparative quantification is carried out among the colorectal tissues or colorectal benign tumor tissues of a healthy subject, the tissues of a non-metastatic colorectal cancer case, and the tissues of a metastatic colorectal cancer case, so that proteins found to fluctuate among the aforementioned tissues can be narrowed as candidate proteins. Moreover, such biomarker candidate proteins can also be searched by using proteins that have been reported to be associated with the onset or progression of colorectal cancer according to previous studies. A fluctuation in the expression levels among the tissues can be verified according to a target proteomics method involving a SRM/MRM (selected reaction monitoring/multiple reaction monitoring) method (Gillette M A et al., Nat Methods. 2013; 10: 28-34), by which, with respect to such biomarker candidates, parent ions having a specific mass are destroyed, and specific ions in the generated daughter ions are then detected, so that a peptide derived from a target protein can be detected with high sensitivity in a complicated sample. According to this verification, final candidates of colorectal cancer biomarkers can be selected. Subsequently, whether or not these biomarker candidates can be detected and quantified in a biological sample such as blood is confirmed. During this operation, the biomarker candidates are preferably detected and quantified in an extracellular vesicle (EV) fraction in blood.

In the method of the present invention, the following proteins may be considered to be biomarkers for detection of colorectal cancer:

### 1. Annexin A11 (ANXA 11) (SEQ ID NO: 1);

### 2. Annexin A3 (ANXA 3) (SEQ ID NO: 2);

### 3. Annexin A4 (ANXA 4) (SEQ ID NO: 3);

### 4. Tanascin-N(TNN) (SEQ ID NO: 4);

5. Transferrin receptor protein 1 (TFRC) (SEQ ID NO: 5);
6. Glucose transporter 1(GLUT-1) (SLC2A1) (SEQ ID NO: 6);
7. Complement component C9 (C9) (SEQ ID NO: 7);
8. CD88 antigen (C5AR1) (SEQ ID NO: 8);
9. 78 kDa glucose-regulated protein (HSPA5) (SEQ ID NO: 9);
10. α-1-acid glycoprotein (ORM1) (SEQ ID NO: 10);
11. Matrix metalloproteinase-9 (MMP9) (SEQ ID NO: 11);

### 12. Angiopoietin-1 (ANGPT1) (SEQ ID NO: 12);

13. CD67 antigen (CEACAM8) (SEQ ID NO: 13);

### 14. Mucin-5B (MUC5B) (SEQ ID NO: 14);

15. Adapter protein GRB2 (GRB2) (SEQ ID NO: 15);

### 16. Annexin A5(Annexin A5)(ANXA 5) (SEQ ID NO: 16);

### 17. Olfactomedin-4 (OLFM4) (SEQ ID NO: 17);

18. Neutral amino acid transporter B(0) (SLC1A5) (SEQ ID NO: 18);

### 19. Tripeptidyl-peptidase 1 (TPP1) (SEQ ID NO: 19);

20. Heat shock-related 70 kDa protein 2 (HSPA2) (SEQ ID NO: 20);
21. Proteasome subunit α type-5 (PSMA5) (SEQ ID NO: 21); and
22. Neutrophil gelatinase-associated lipocalin (LCN2) (SEQ ID NO: 22).

Moreover, examples of the partial peptides of the above-described proteins that can be used as biomarkers for detection of colorectal cancer may include:

a peptide consisting of the amino acid sequence as set forth in SEQ ID NO: 23 or 24 (a partial peptide of Annexin A11),
a peptide consisting of the amino acid sequence as set forth in SEQ ID NO: 25 or 26 (a partial peptide of Annexin A3),
a peptide consisting of the amino acid sequence as set forth in SEQ ID NO: 27 or 28 (a partial peptide of Annexin A4),
a peptide consisting of the amino acid sequence as set forth in SEQ ID NO: 29 or 30 (a partial peptide of Tanascin-N),
a peptide consisting of the amino acid sequence as set forth in SEQ ID NO: 31 or 32 (a partial peptide of Transferrin receptor protein 1),
a peptide consisting of the amino acid sequence as set forth in SEQ ID NO: 33 or 34 (a partial peptide of Glucose transporter 1),
a peptide consisting of the amino acid sequence as set forth in SEQ ID NO: 35 or 36 (a partial peptide of Complement component C9),
a peptide consisting of the amino acid sequence as set forth in SEQ ID NO: 37 or 38 (a partial peptide of CD88 antigen),
a peptide consisting of the amino acid sequence as set forth in SEQ ID NO: 39 or 40 (a partial peptide of 78 kDa glucose-regulated protein),
a peptide consisting of the amino acid sequence as set forth in SEQ ID NO: 41 or 42 (a partial peptide of α-1-acid glycoprotein),
a peptide consisting of the amino acid sequence as set forth in SEQ ID NO: 43 or 44 (a partial peptide of Matrix metalloproteinase-9),
a peptide consisting of the amino acid sequence as set forth in SEQ ID NO: 45 or 46 (a partial peptide of Angiopoietin-1),
a peptide consisting of the amino acid sequence as set forth in SEQ ID NO: 47 or 48 (a partial peptide of CD67 antigen),
a peptide consisting of the amino acid sequence as set forth in SEQ ID NO: 49 or 50 (a partial peptide of Mucin-5B),
a peptide consisting of the amino acid sequence as set forth in SEQ ID NO: 51 or 52 (a partial peptide of Adapter protein GRB2),
a peptide consisting of the amino acid sequence as set forth in SEQ ID NO: 53 (a partial peptide of Annexin A5),
a peptide consisting of the amino acid sequence as set forth in SEQ ID NO: 54 (a partial peptide of Olfactomedin-4),
a peptide consisting of the amino acid sequence as set forth in SEQ ID NO: 55 (a partial peptide of Neutral amino acid transporter B(0)),
a peptide consisting of the amino acid sequence as set forth in SEQ ID NO: 56 (a partial peptide of Tripeptidyl-peptidase 1),
a peptide consisting of the amino acid sequence as set forth in SEQ ID NO: 57 (a partial peptide of Heat shock-related 70 kDa protein 2),
a peptide consisting of the amino acid sequence as set forth in SEQ ID NO: 58 (a partial peptide of Proteasome subunit α type-5), and
a peptide consisting of the amino acid sequence as set forth in SEQ ID NO: 59 (a partial peptide of Neutrophil gelatinase-associated lipocalin).

In the present invention, the above-described proteins, or partial peptides of the proteins are used as biomarkers. The above-described proteins or partial peptides thereof may also include proteins each consisting of an amino acid sequence comprising a deletion, substitution or addition of one or several amino acids in the amino acid sequences as set forth in SEQ ID NOS: 1 to 59, or peptides thereof. These proteins or peptides can also be used as biomarkers in the method of the present invention. Herein, the phrase “one or several” means “1 or 3,” “1 or 2,” or “1.”

The term “peptide” generally means amino acid residues having a molecular weight of 10,000 or less, which are connected with one another via a peptide bond. The number of amino acid residues is from several to approximately 50 or less. In the present description, the protein or a partial peptide thereof can be used as a biomarker. When the partial peptide is used as a biomarker, it is a peptide having a partial amino acid sequence that is a part of the amino acid sequence of a protein, and the molecular weight thereof is not limited. It is preferably a peptide having a molecular weight of 10,000 or less. There is a case where the partial peptide is generated in the expression and synthesis process involving transcription and/or translation. There is also a case where, after synthesis of a protein, the protein is subjected to digestive degradation in vivo, so that the partial peptide is generated as a digestive degradation product peptide.

As a result of the ROC analysis, it has been revealed that, among these proteins and peptides, Glucose transporter 1, Transferrin receptor protein 1, Annexin A5, Matrix metalloproteinase-9, Annexin A4, Annexin A11, Tanascin-N, Annexin A3, Olfactomedin-4, CD88 antigen, Neutrophil gelatinase-associated lipocalin or tripeptidyl-peptidase 1, or their partial peptides can detect colorectal cancer with favorable detection sensitivity and specificity. Thus, the aforementioned proteins, peptides or their partial peptides are preferably used as biomarkers for detection of colorectal cancer. Furthermore, Annexin A4 or Annexin A11, or their partial peptides are more preferably used as biomarkers for detection of colorectal cancer.

In the present invention, among the above-described 22 proteins and partial peptides, at least one protein and/or at least one partial peptide can be used as a biomarker. However, preferably 2 types or more, more preferably 3 types or more, 4 types or more, 5 types or more, 6 types or more, 7 types or more, 8 types or more, 9 types or more, 10 types or more, 11 types or more, 12 types or more, 13 types or more, 14 types or more, 15 types or more, 16 types or more, 17 types or more, 18 types or more, 19 types or more, 20 types or more, 21 types or more, or 22 types proteins or partial peptides thereof can be used to detect colorectal cancer with higher accuracy. In this case, it may be a combination of different proteins, or may also be a combination of the partial peptide of a certain protein and the partial peptide of a protein other than the certain protein, or a combination of partial peptides. Otherwise, a plurality of intact proteins alone may be used as biomarkers. Thus, by using a plurality of proteins or partial peptides as biomarkers, colorectal cancer can be more accurately detected, and also, the progression of colorectal cancer can be precisely judged.

Examples of the combination of proteins may include a combination of Glucose transporter 1 and Transferrin receptor protein 1, a combination of Glucose transporter 1 and Angiopoietin-1, a combination of Matrix metalloproteinase-9 and Transferrin receptor protein 1, a combination of Transferrin receptor protein 1 and Neutrophil gelatinase-associated lipocalin, a combination of Transferrin receptor protein 1 and Angiopoietin-1, a combination of Transferrin receptor protein 1 and Adapter protein, a combination of 78 kDa glucose-regulated protein and Tripeptidyl-peptidase 1, a combination of Tanascin-N and Neutrophil gelatinase-associated lipocalin, a combination of Transferrin receptor protein 1, Neutrophil gelatinase-associated lipocalin and Angiopoietin-1, a combination of Transferrin receptor protein 1 and Olfactomedin-4, a combination of Tanascin-N and Angiopoietin-1, a combination of Olfactomedin-4 and Angiopoietin-1, a combination of Tanascin-N and Olfactomedin-4, and a combination of Olfactomedin-4 and Tripeptidyl-peptidase 1. Combination of the partial peptides of the aforementioned respective proteins may also be adequate.

Further, the above-described protein or a partial peptide thereof is combined with CEA (Carcinoembryonic antigen) that has conventionally been known as a biomarker for colorectal cancer, and the combination is used as a biomarker for detection of colorectal cancer, so that colorectal cancer can be detected with higher sensitivity and higher specificity than in the case of a single use thereof.

The expression of the above-described protein or a partial peptide thereof increases in colorectal cancer patients. Accordingly, the protein or a partial peptide thereof in blood may be quantified. Herein, the blood includes serum and plasma. Moreover, the protein is present in an extracellular vesicle (EV). As such, EV present in blood is isolated, and is then concentrated, as necessary, and thereafter, the protein or a partial peptide thereof may be detected in the EV. EV can be separated as an EV fraction, for example, according to ultracentrifugation. Otherwise, EV can be isolated using a marker specific to the EV. Examples of such an EV marker may include CD9, CD63, and CD81, and EV can be separated by utilizing magnetic beads to which antibodies reacting against these markers bind, etc. Another example of the EV marker may be phosphatidylserine (PS), and EV can be separated by utilizing magnetic beads to which phosphatidylserine-binding proteins having affinity for phosphatidylserine bind, etc. Examples of the phosphatidylserine-binding protein may include a T-cell immunoglobulin and mucin domain-containing molecule 4 (Tim4), Annexin A5, Annexin V, and milk fat globule-EGF factor protein 8 (MFG-E8).

By the method of the present invention, protein/partial peptide profile in a biological sample is examined, but the biological sample used herein is not limited. Examples of the biological sample may include liquid samples that can be collected from living bodies, including body fluids such as whole blood, serum, plasma, urine, and saliva. Besides, a protein, a partial protein, and a partial peptide in such a biological sample may be further degraded depending on the preserved state of the biological sample. Thus, the biological sample is preferably used without repeating freezing and thawing operations. Moreover, a protease inhibitor and the like may be added to the biological sample. Otherwise, exosome may be separated with the above-described marker as an indicator, using FACS or a flow cytometer.

A protein or a peptide may be extracted from the isolated exosome, and the protein or the peptide may be then measured.

A protein or a partial peptide can be detected according to various methods. Examples of such various methods are given below, but are not limited thereto.

For example, a protein or a partial peptide thereof to be detected can be measured according to an immunoassay using an antibody reacting against such a protein or a partial peptide thereof to be detected. Examples of the immunoassay may include solid phase immunoassays (RIA, EIA, FIA, CLIA, etc.), a dot blotting method, a latex agglutination method (LA: Latex Agglutination-Turbidimetric Immunoassay), and an immunochromatography method. The antibody can be immobilized on a substrate and can be then used.

Among these methods, from the viewpoint of quantitativity, an ELISA (Enzyme-Linked ImmunoSorbent Assay) method that is one type of EIA (Enzyme Immunoassay) method is preferable. According to the ELISA method, a specimen is added into a well of a microtiter plate, on which an antibody is immobilized, to perform an antigen-antibody reaction, and thereafter, an enzyme-labeled antibody is further added thereto to perform an antigen-antibody reaction, followed by washing. Thereafter, the reaction mixture is allowed to react with and/or develop color with an enzyme substrate, the absorbance is measured, and a marker protein or a partial peptide thereof in the sample is detected, and thereafter, the concentration of the protein or a partial peptide thereof in the specimen can be calculated from the measured value. Otherwise, using a fluorescently-labeled antibody, an antigen-antibody reaction may be performed, and the fluorescence may be then measured. The antigen-antibody reaction can be carried out at 4° C. to 45° C., more preferably at 20° C. to 40° C., and further preferably at 25° C. to 38° C. In addition, the reaction time is approximately 10 minutes to 18 hours, more preferably approximately 10 minutes to 1 hour, and further preferably approximately 30 minutes to 1 hour.

The antibody reacting against a marker protein or a partial peptide thereof used in immunoassays may be either a monoclonal antibody or a polyclonal antibody, and further, binding active fragments of monoclonal antibodies, such as Fab, F(ab′), and F(ab′)₂ can also be used.

The present invention also includes a test reagent or kit for detecting colorectal cancer, comprising antibodies reacting against one or more of the above-described 22 types of proteins or partial peptides thereof.

A biomarker protein for detection of colorectal cancer or a partial peptide thereof can also be analyzed according to mass spectrometry using a mass spectrometer. Such mass spectrometry is particularly suitable for detection of a partial peptide.

Moreover, a biomarker protein or a partial peptide thereof can also be detected using a mass spectrometer. In particular, for detection of a partial peptide of a biomarker protein, the use of a mass spectrometer is suitable. The mass spectrometer comprises a sample introduction part, an ionization chamber, an analysis part, a detection part, a record part, and the like. Examples of the ionization method that may be applied herein may include an electron ionization (EI) method, a chemical ionization (CI) method, a field desorption (FD) method, a secondary ion mass spectrometry (SIMS) method, a fast atom bombardment (FAB) method, a matrix-assisted laser desorption ionization (MALDI) method, and an electrospray ionization (ESI) method. Moreover, for the analysis part, a double-focusing mass spectrometer, a quadrupole mass spectrometer, a time-of-flight mass spectrometer, a Fourier transform mass spectrometer, an ion cyclotron mass spectrometer, or the like is used. For performing a precise analysis, a tandem mass spectrometer (MS/MS), in which two mass spectrometers are connected with each other, can also be used.

The mass spectrometer may be used alone, or may also be connected with separation equipment such as liquid chromatography, measurement equipment, and the like, and thus, a liquid chromatograph-mass spectrometry (LC/MS or LC/MS/MS), in which mass spectrometry is combined with high performance liquid chromatography, can be used in the analysis. Mass spectrometry can be carried out by selected reaction monitoring (SRM) or multiple reaction monitoring (MRM) using a triple quadrupole mass spectrometer, according to an LC/MS (LC/MS/MS) System that is broadly used for quantitative detection of a peptide in the present technical field. According to SRM/MRM, many factors can be simultaneously measured, and thus, several hundreds of proteins or peptides can be simultaneously measured by a single measurement.

When a large amount of the above described biomarker protein or a partial peptide thereof is present in a sample collected from a subject, namely, when the subject is positive, the subject can be determined to be affected with colorectal cancer.

In the present invention, a sample collected from a healthy subject may be simultaneously measured as a negative control. In this case, when a subject is affected with colorectal cancer, the concentration of a biomarker protein or a partial peptide thereof in the specimen of the subject increases in comparison to the healthy subject. Hence, when the concentration of the biomarker protein or a partial peptide thereof in the subject is higher than that in the healthy subject, the biomarker protein or a partial peptide thereof is determined to be positive, and thus, the subject can be determined to be affected with colorectal cancer. With regard to whether or not the biomarker protein or a partial peptide thereof in a sample is positive, a cutoff value has previously been determined with respect to the ratio between the peak area value of a biomarker peptide that has previously been detected according to the SRM/MRM method and the peak area value of a stable isotope-labeled peptide used as an internal standard, or with respect to an antibody measured value, and the cutoff value is used as a reference. When the measured value exceeds the cutoff value, the biomarker protein or a partial peptide thereof can be determined to be positive.

The cutoff value can be determined, for example, by ROC (receiver operating characteristic curve) analysis. Moreover, the diagnostic accuracy (sensitivity and specificity) of the method of the present invention can be determined by the ROC analysis. According to the ROC analysis, the biomarker protein or a partial peptide thereof in a sample collected from a colorectal cancer patient and the biomarker protein or a partial peptide thereof in a sample collected from a healthy subject are measured, and sensitivity and false positive rate (1-specificity) are then calculated for each cutoff value. Then, the horizontal axis is set to be 1-specificity, the longitudinal axis is set to be sensitivity, and the values are plotted on the coordinates.

When diagnostic accuracy is analyzed based on the measurement results of the method of the present invention according to the ROC analysis, the area under the curve (AUC) is high (0.9 or more); the sensitivity is 70% or more, preferably 80% or more, more preferably 85% or more, and further preferably 90% or more; and the specificity is 70% or more, preferably 75% or more, and more preferably 80% or more. According to the method of the present invention, early-stage colorectal cancer can be detected with extremely high accuracy.

### EXAMPLES

The present invention will be specifically described in the following examples. However, these examples are not intended to limit the scope of the present invention.

### Methods

Serums of colorectal cancer patients and healthy subjects, and colorectal cancer cultured cells Serum was collected from 51 colorectal cancer patients and 26 healthy subjects (collected at School of Medicine, Chiba University). Each specimen serum sample was preserved at −80° C. before use in analysis.

Four colorectal cancer cell lines, namely, HCT116 (ATCC; CCL-247), DLD-1 (ATCC; CCL-221), SW480 (ATCC; CCL-228), and SW620 (ATCC; CCL-227) were cultured in an RPMI-1640 medium (Gibco Laboratories) supplemented with 10% fetal bovine serum (FBS) and antibiotics.

The cells of each line were cultured in a 5% CO₂ incubator, while the temperature was maintained at 37° C., until the cells have grown and have become sub-confluent. Thereafter, the cultured cells were washed with a FBS-free medium, and a fresh FBS-free medium was then added thereto. The thus obtained cells were further cultured in the incubator for 48 hours. Thereafter, the conditioned supernatant medium was gathered and was used as a sample for use in isolation of EV (extracellular vesicles).

### Isolation of EV (Extracellular Vesicles)

Isolation of EV was carried out according to an ultracentrifugation method. A sucrose buffer was placed on the bottom of a centrifuge tube, so that low-density contaminants can be efficiently separated upon the recovery of EV. As a treatment performed before ultracentrifugation, 100 μl of the serum was centrifuged at 300 g for 10 minutes to remove large contaminants. Thereafter, the recovered serum was passed through a 0.22-μm spin filter (Agilent Technologies, Santa Clara, Calif.), and was then centrifuged with a 30% sucrose/D₂O buffer at 100,000 g for 90 minutes. The buffer was recovered, and was further subjected to an ultracentrifuge at 100,000 g for 70 minutes twice, and an EV fraction deposited on the centrifuge tube was then recovered.

Extraction of Protein from EV and Digestion Thereof

Extraction of a protein and the subsequent digestion thereof were carried out in accordance with phase transfer surfactant (PTS) protocols (Masuda T et al. J. Proteome Res. 7, 731-740 (2008)). The EV fraction protein was lysed using an MPEX PTS reagent kit (GL Science, Japan), and dithiothreitol was then added thereto to a final concentration of 5 mM. The thus obtained mixture was subjected to a reduction reaction at room temperature for 30 minutes, and iodoacetamide was further added to the reaction mixture to a final concentration of 20 mM, followed by an alkylation reaction.

Thereafter, 1% (w/w) trypsin (proteomics grade; Roche Mannheim, Germany) was added to the sample, and the obtained mixture was digested at 37° C. overnight. After completion of the digestion, ethyl acetate in an amount equal to the liquid amount and trifluoroacetic acid (final concentration: 1%) were added to the resultant, and the obtained mixture was then stirred using Vortex, so that the surfactant contained in the liquid was separated into an organic layer. After completion of the centrifugation, a water phase containing the peptide was recovered, and desalination was then carried out using Stage Tips (Rappsilber J. et al., Nat. Protoc. 2, 1896-1906 (2007)).

### Liquid Chromatography (LC)-Mass Spectrometer (MS/MS) and Data Analysis of Proteome

The digested peptide was fractionated into 7 fractions, using a C18-SCX StageTip chromatography column. Thereafter, analysis was carried out using a mass spectrometer (Q-Exactive (Thermo Scientific, Bremen, Germany)) connected with LC (UltiMate3000 Nanoflow HPLC system (Dionex, Sunnyvale, Calif.)). The sample was introduced into the mass spectrometer by using the present inventors' own making analysis column, in which a 1.9-μm C18-AQ resin was enclosed in a needle with an inner diameter of 75 μm and a length of 300 mm. The mobile phase of LC was composed of buffer A (0.1% formic acid and 2% acetonitrile) and buffer B (0.1% formic acid and 90% acetonitrile). The digested peptide was dissolved in the buffer A, and was then loaded on a trap column (0.075×20 mm, Acclaim PepMap RSLC Nano-Trap Column; Thermo Scientific). Regarding Nano LC, the solution was supplied at a rate of 280 nL/min, and the mobile phase was developed with a gradient from 5% to 35% buffer B for 120 minutes. Regarding mass spectrometry, the measurement was carried out with Full MS scan (350 to 1800m/z, ion integration: 3×10⁶, resolution: 70,000) and MS/MS scan (Top10 precursor ion, injection time: 120 ms, resolution: 35,000, MS/MS ion selection threshold: 5×10⁴ counts, separation width: 3.0 Da). The data file was analyzed using MaxQuant software (Ver 1.5.1.2). Andromeda was used as a search engine, and a peak list was searched against UniProt human protein database. The precursor mass error range was set to be 7 ppm, and the fragment ion mass error range was set to be 0.01 Da. The identified protein and peptide were determined with a false positive rate of less than 1% (FDR 1%>) with respect to the reverse database.

### LC-SRM/MRM Analysis

LC-SRM/MRM analysis was carried out according to the previously reported method (Kume, H. et al., Mol. Cell. Proteomics 13, 1471-1484 (2014): Muraoka, S. et al., J. Proteome Res. 11, 4201-4210 (2012): Narumi, R. et al., J. Proteome Res. 11, 5311-5322 (2012)). The digested peptide was dissolved in a 2% acetonitrile solution containing 0.1% trifluoroacetic acid (TFA), and thereafter, it was analysis by using a triple quadrupole mass spectrometer TSQ-Vantage (Thermo Fisher Scientific, Bremen, Germany) connected with NanoFlow LC Paradigm MS2 (Michrom BioResources, Auburn, Calif.). The sample was introduced into the mass spectrometer by using the present inventors' own making analysis column, in which a 1.9-μm C18-AQ resin was enclosed in a needle with an inner diameter of 75 μm and a length of 100 mm. The mobile phase of LC was composed of buffer A (0.1% formic acid and 2% acetonitrile) and buffer B (0.1% formic acid and 90% acetonitrile). The digested peptide was dissolved in the buffer A, and was then loaded on a trap column (0.075×20 mm, Acclaim PepMap RSLC Nano-Trap Column; Thermo Scientific). Regarding Nano LC, the solution was supplied at a rate of 280 nL/min, and the mobile phase was developed with a gradient from 5% to 35% buffer B for 60 minutes. The analysis was carried out at an SRM mode under the following conditions (Q1 Peak Width 0.7 FWHM; Cycle time: 1 sec; and Collision Gass Pressure 1.8 mTorr). The collision energy was optimized for every SRM transition, and the intensity of each transition was measured at a schedule mode of a peak time width of 5 minutes.

### Quantification of Target Peptide Using SI-Peptide

With regard to quantification of a target peptide according to SRM, a stable isotope-labeled peptide (SI-peptide) having the same sequence as that of the target peptide was mixed as an internal standard with EV prepared from each specimen serum, and thereafter, the amount was calculated based on the peak area ratio of the endogenous peptide/the SI-peptide detected according to the SRM analysis. The spike amount of the SI-peptide having the same sequence as that of the target peptide was adjusted to be close to that of the endogenous peptide by performing a pre-analysis.

### Statistical Analysis of Peak Area Ratio According to SRM Analysis

In order to evaluate the target peptide as a colorectal biomarker based on the peak area ratio of each specimen calculated according to the SRM analysis, the statistical analysis of the SRM data was carried out. For the statistical analysis, SPSS software (Ver. 23) (SPSS Inc., Chicago, Ill.) was used. First, regarding the evaluation of a single target peptide as a biomarker, the value of AUC (Area under the Curve) was calculated according to the ROC analysis (ROC: Receiver Operating Characteristic). Furthermore, a plurality of target peptides were combined with one another, and the thus combined target peptides were also evaluated as multiple markers. For the combination of the target peptides, a logistic regression model was prepared using SPSS software, and was then evaluated. A combination of peptides, for which the model was effective, was also evaluated with the value of AUC calculated according to the ROC analysis.

### Results

### Searching for CRC-Related Biomarker Candidate Proteins

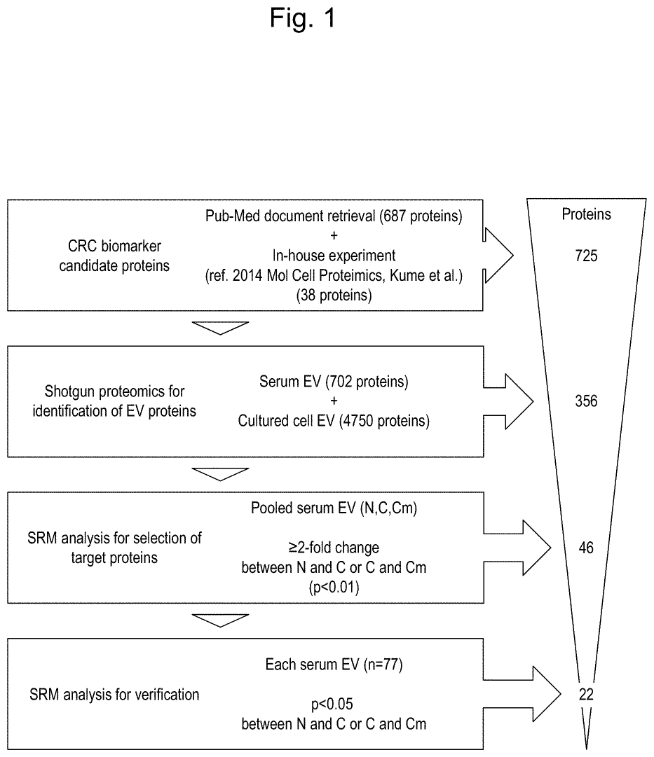

FIG. 1 shows a strategy for searching for the biomarker candidates of the present case. A list of CRC biomarker candidate proteins was acquired from PubMed Database regarding medical and biological publications. As a retrieval style, “cancer” AND “colorectal” AND “expression” was used, and document retrieval was performed on the database from 2003 to 2014, and as a result, 687 colorectal cancer-related proteins were listed up as CRC biomarker candidates. The candidate proteins were selected in accordance with the following criteria (1) to (4).

(1) The expression of a protein in human CRC tissues or blood has been confirmed according to Western blot, ELISA, or immunohistochemistry.
(2) An increase in the expression of a target protein has been confirmed in cancer. (Since a protein with an increased expression is detected more easily than a protein with a decreased expression, it is suitable as a biomarker.)
(3) The molecular function of a target protein relevant to the expression and progression of cancer has been experimentally confirmed by RNAi or the overexpression of molecules.
(4) Proteins specified only by large-scale analysis (e.g., omics analysis) have been excluded.

Moreover, the present inventors had previously specified 44 CRC biomarker candidate proteins by performing targeted proteomics on clinical specimens (Kume, H. et al., Mol. Cell. Proteomics 13, 1471-1484 (2014)). These 44 proteins were gathered with the above-described biomarker candidates, and then, overlapped proteins were excluded therefrom. The thus obtained total 725 proteins were selected as CRC biomarker candidates.

### Selection of Biomarker Candidates Contained in EV According to Shotgun Proteomics Analysis

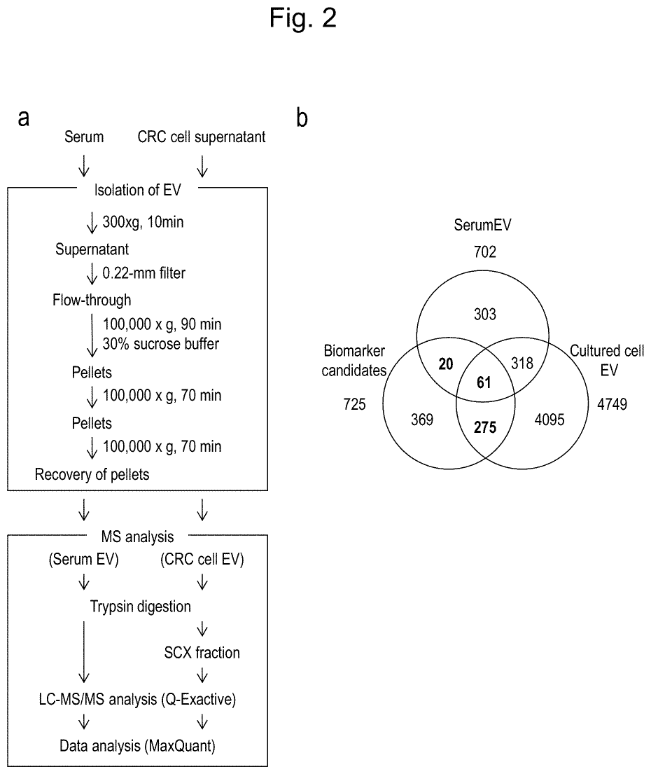

In order to select proteins present in EV from the selected biomarker candidate proteins, EV prepared from serum and cultured cell supernatant was used, and the qualitative analysis of EV proteins was carried out according to shotgun proteomics analysis. A work flow of the shotgun proteomics is shown in FIG. 2a.

Preparation of EV from serum and cell supernatant was carried out according to an ultracentrifugation method. Serum EV was collected from 8 healthy subjects, 8 non-metastatic cancer patients, and 8 metastatic cancer patients. A cultured cell supernatant was collected from each of 4 types of CRC cells (HCT116, DLD-1, SW480, and SW620). The extracted proteins were digested according to a phase transfer solubilizer (PTS) method, and were then fractionated using a C-18 Stage SCX Tip column (Masuda, T et al., J. Proteome Res. 7, 731-740 (2008); Adachi, J. et al. Anal. Chem. 88, 7899-7903 (2016)). The fraction samples were analyzed according to LC-MS/MS, and then, as a result of Mascot database searching, 702 proteins (serum EV) and 4749 proteins (culture supernatant EV) were identified. By comparing these EV identified proteins with the biomarker candidate proteins, 356 proteins were identified as EV protein-containing biomarker candidates (FIG. 2b).

Selection of SRM Target Peptides from Biomarker Candidate Proteins Identified in EV

In order to verify the effectiveness of the candidate proteins as biomarkers, EV fractions collected from CRC patient serum were subjected to the SRM analysis. SRM candidate peptides were selected from the peptides specified by shotgun proteomics. The candidate peptides were selected in accordance with the following criteria (1) to (3).

(1) The identified proteins had peptide sequences specific to proteins. When there were multiple peptides, targets having high intensity were selected according to shotgun analysis.
(2) Peptides having a cleavage mistake with trypsin or an amino acid modification that was not suitable for the SRM analysis (e.g., oxidized methionine, etc.) were excluded.
(3) In the case of too long peptides (>20 amino acids), it was difficult to synthesize a stable isotope-labeled peptide (SI-peptide). Thus, such too long peptides were excluded from target peptides.

Among all of the identified peptides, 3316 peptides (i.e., 346 proteins) satisfied these criteria.

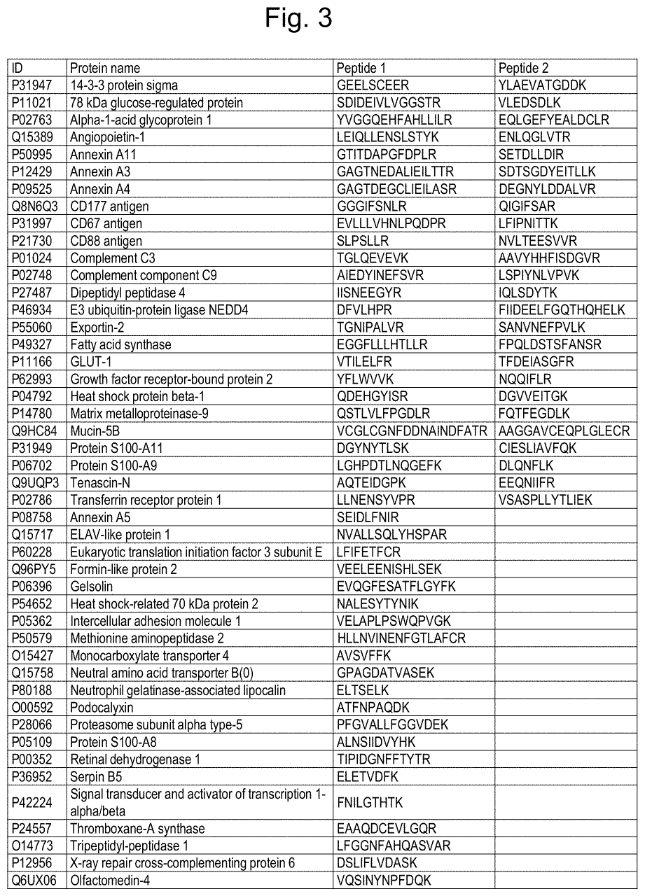

Subsequently, in order to select SRM target peptides from these candidates, EV fractions were prepared from the pooled serum of healthy subjects (N) as non-cancerous controls (N=26), the pooled serum of non-metastatic cancer patients (C) (N=26), and the pooled serum of metastatic cancer patients (Cm) (N=25), and were then used in the SRM analysis. From the results of the shotgun analysis, 3 or 4 transitions (a pair of a precursor ion and a product ion) were selected for a single peptide. In the SRM analysis, the measurement was repeated three times for each pool, and the peptide peak area was quantified using Skyline software. A target peptide, the peak area of which was significantly (2-fold or more) increased (p<0.01) between the pooled samples (between N and C, or between C and Cm), was selected as a marker candidate peptide. Regarding a protein in which two or more candidates peptides were detected, two peptides having high peak intensity according to the SRM analysis were selected. Considering these criteria, 71 peptides (46 proteins) were selected as target peptides serving as biomarker candidates (FIG. 3).

### Quantification of Target Peptides in Serum EV Fraction According to SRM

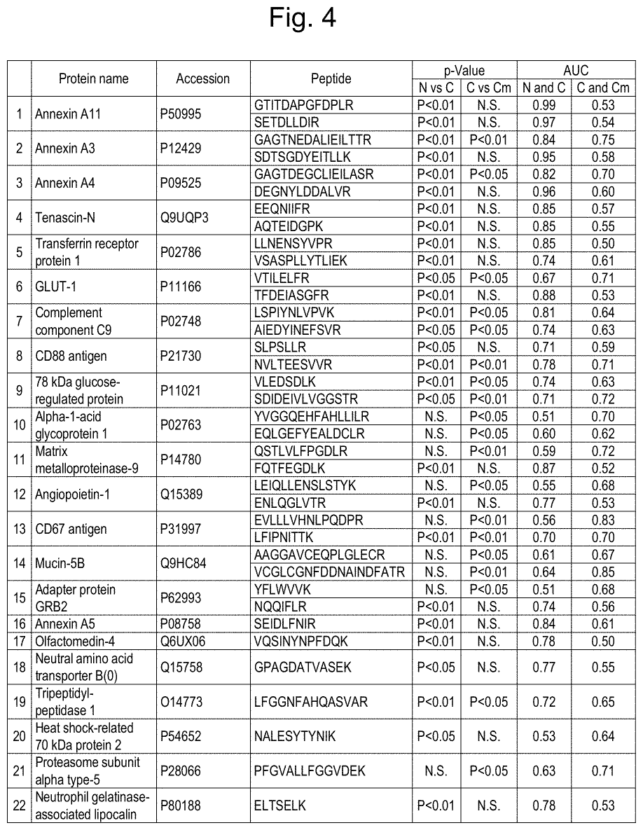

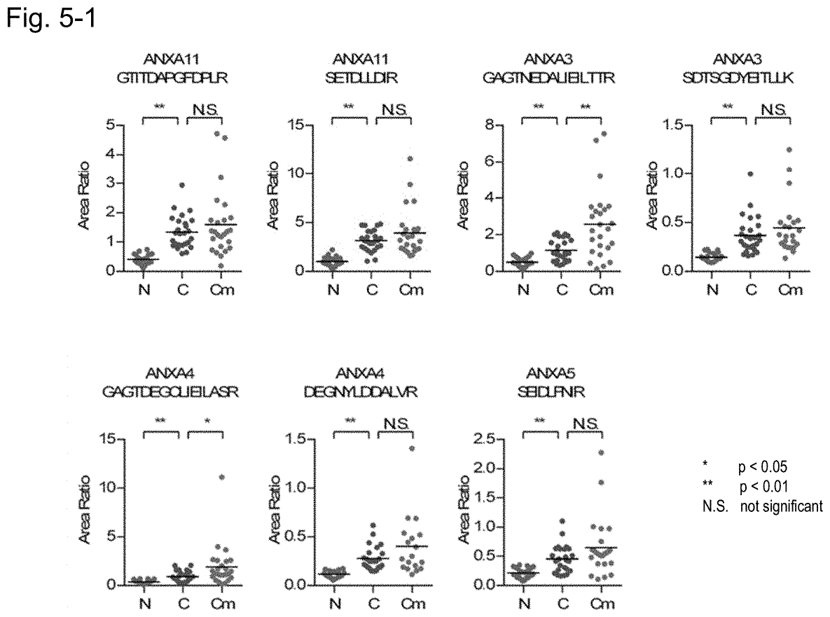

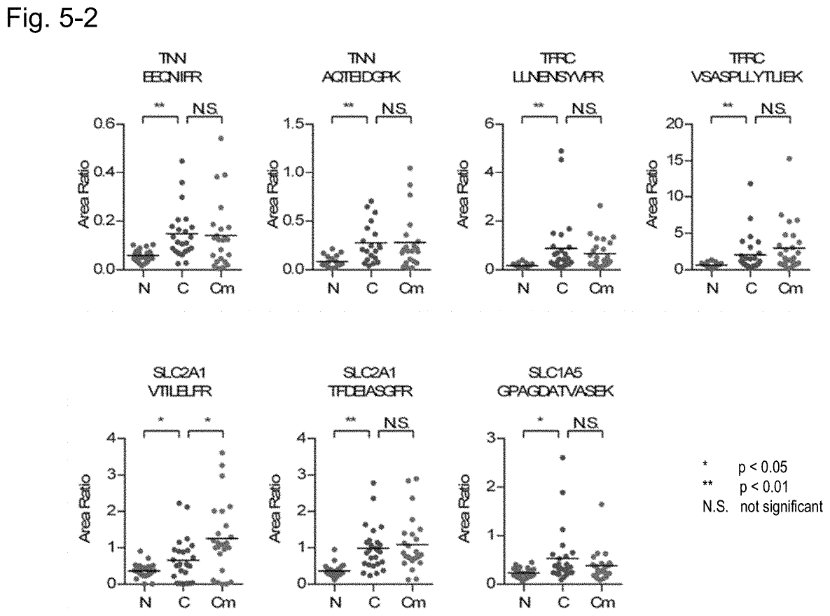

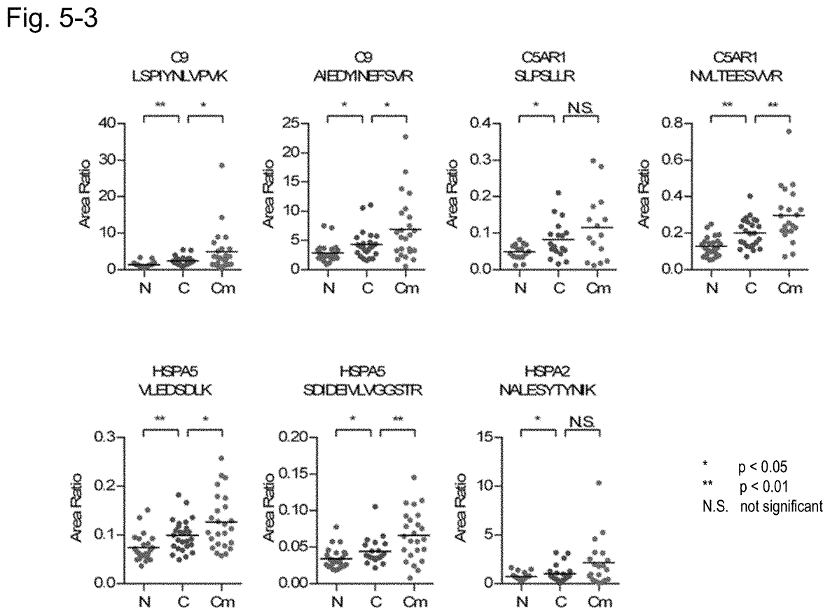

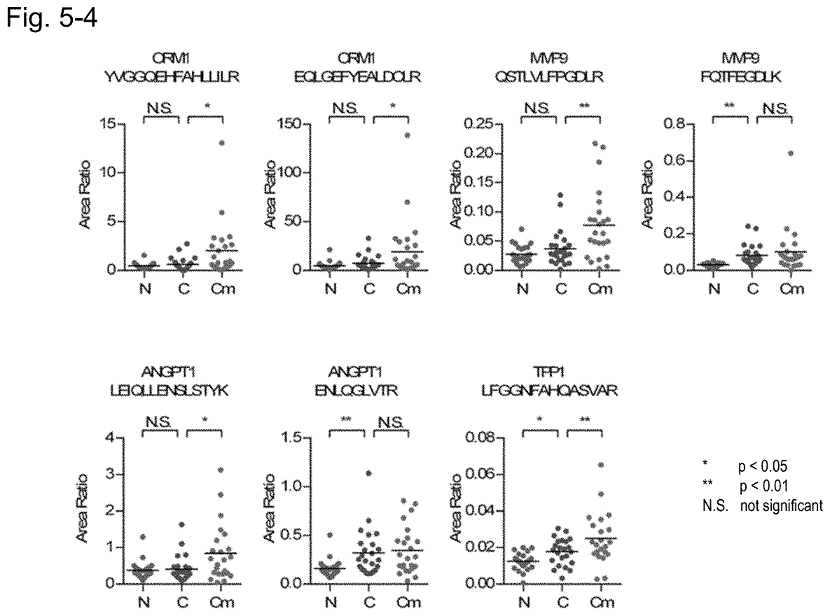

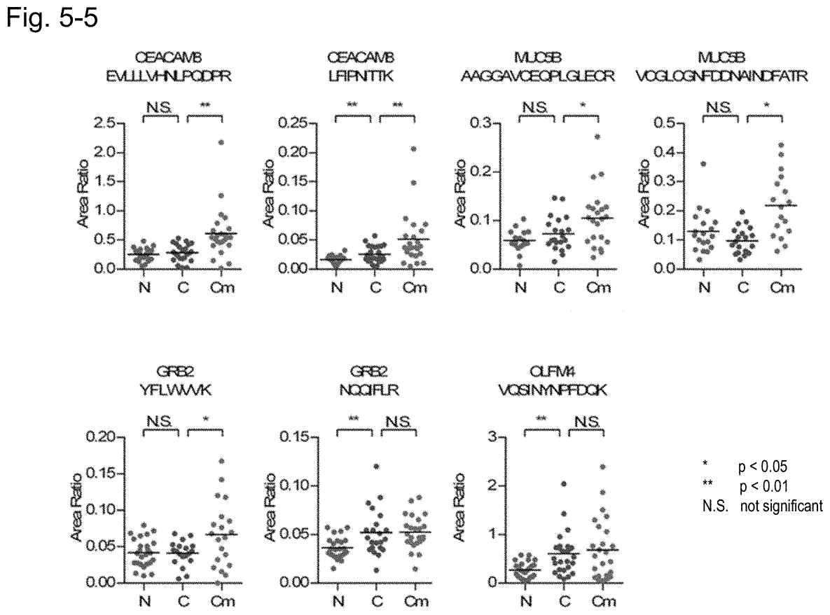

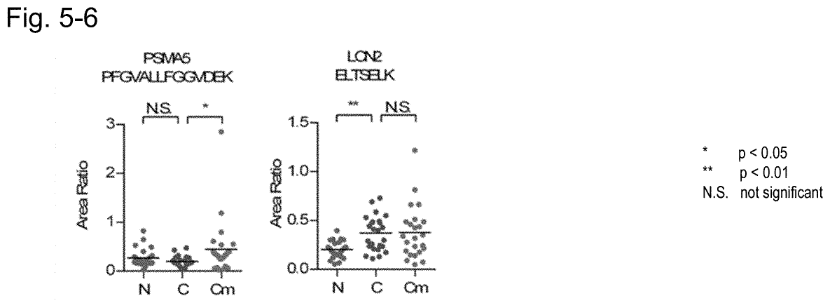

37 Biomarker candidate peptides in the EV fractions prepared from the serums of individual patients were subjected to quantitative analysis according to SRM. Individual EV fractions were prepared from the patient serums of three groups (healthy subjects (N), n=26; non-metastatic cancer patients (C), n=26; and metastatic cancer patients (Cm), n=25). An equal amount of SI-peptide was added as an internal standard to each specimen, and the peak area ratio of the SI-peptide to the endogenous target peptide was calculated using Skyline software. Regarding each target peptide, the quantitative values were compared among the groups N, C, and Cm according to a t-test. As a result, a significant increase in the quantitative values was observed between N and C, or between C and Cm (p<0.05) (FIG. 4 and FIGS. 5-1, 5-2, 5-3, 5-4, 5-5 and 5-6).

### Evaluation of Target Peptides as Biomarkers According to Statistical Analysis

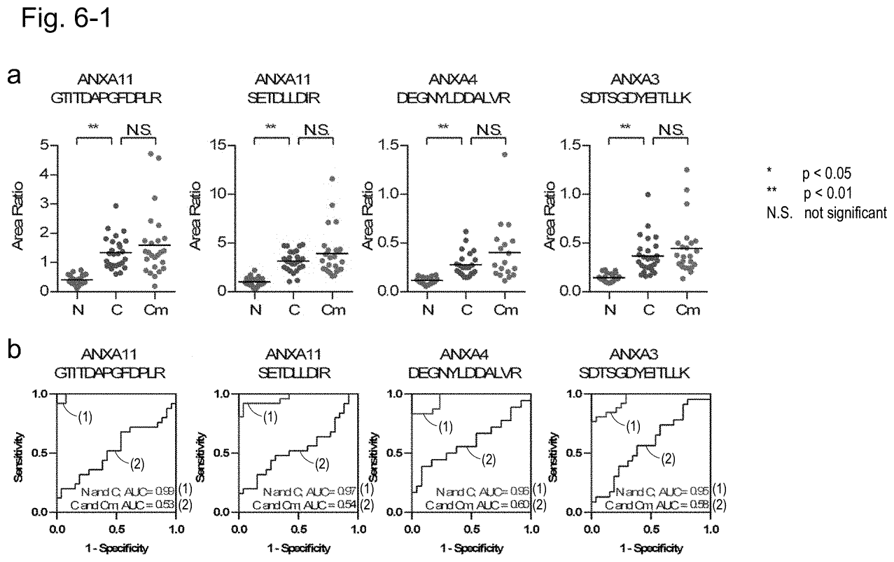

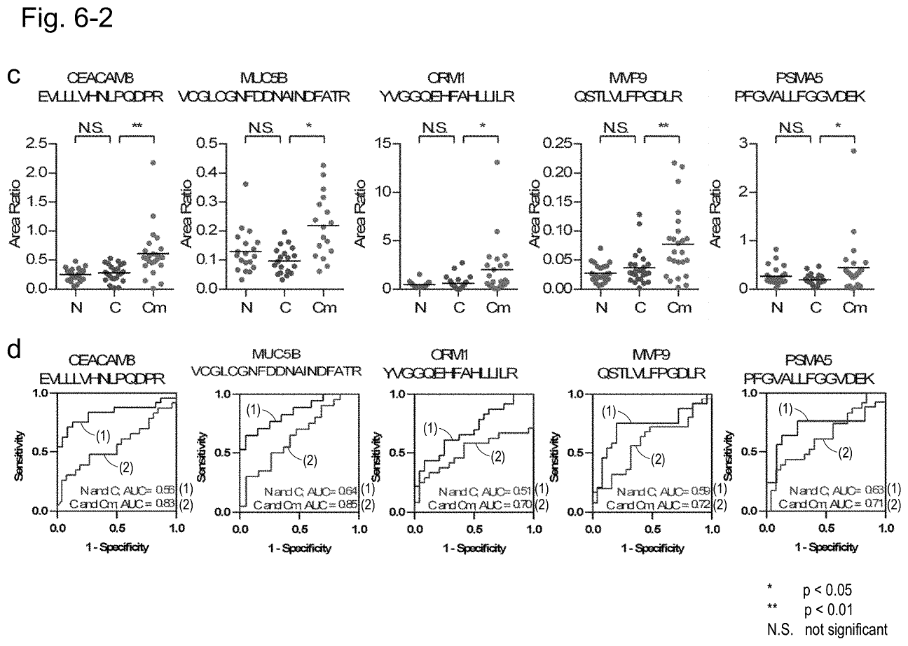

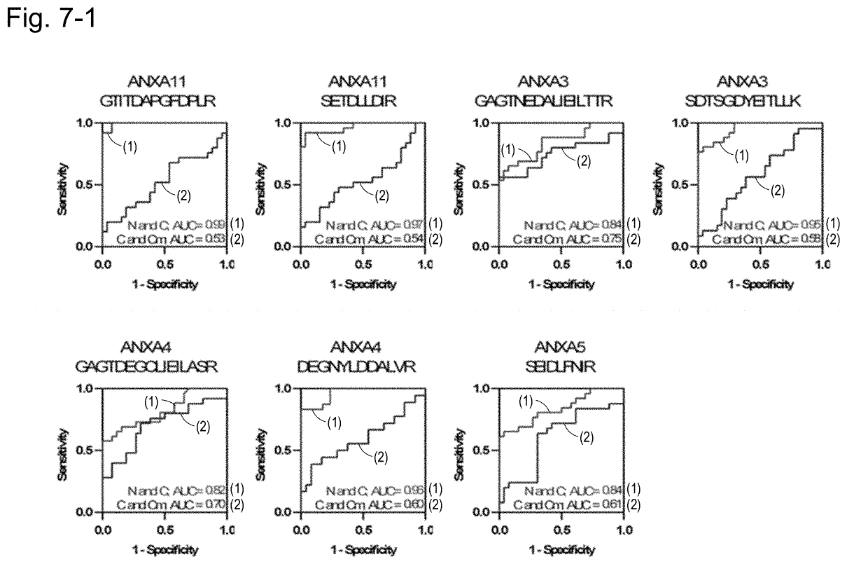

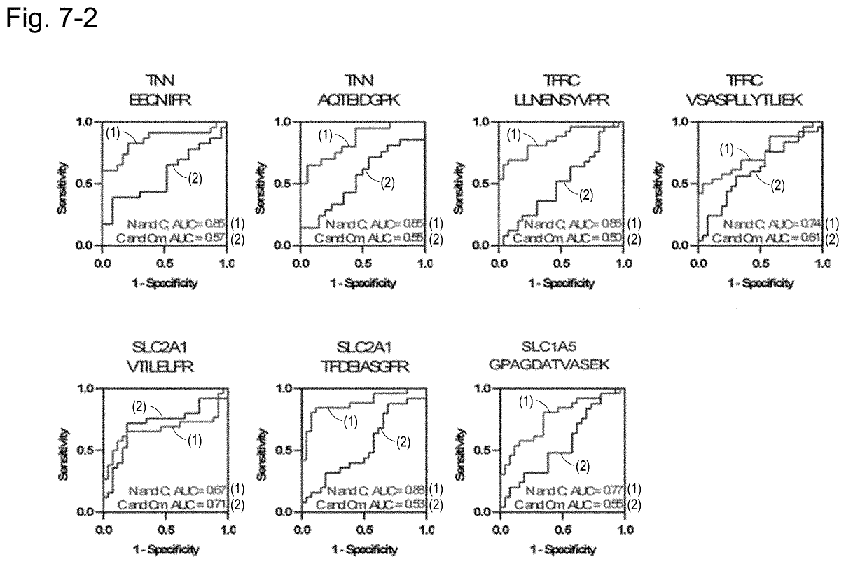

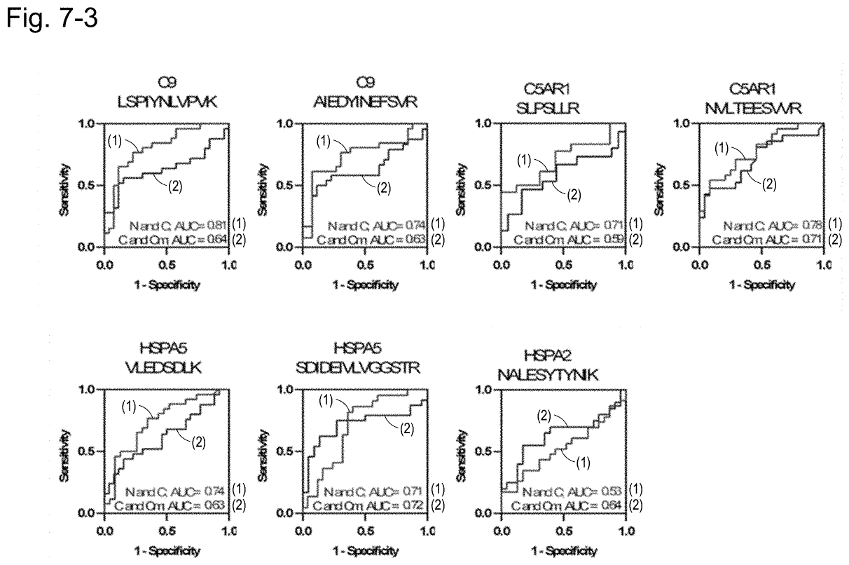

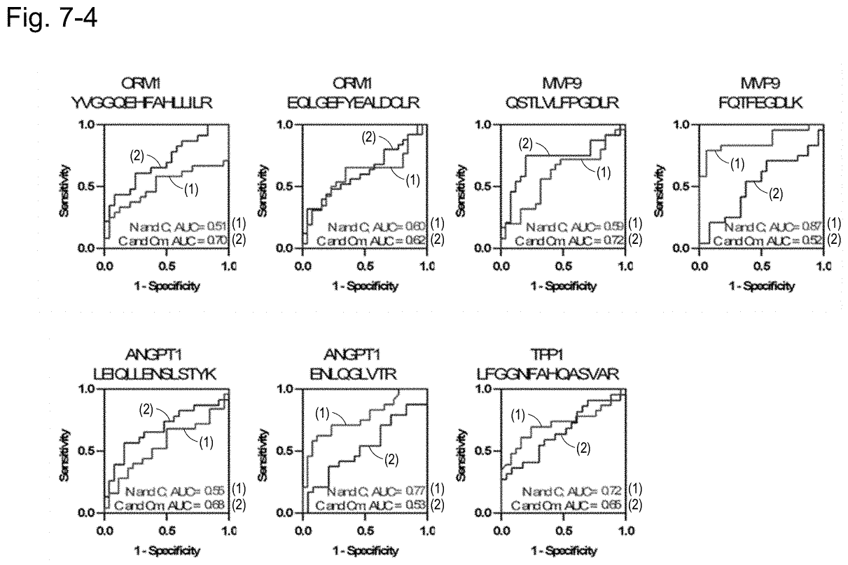

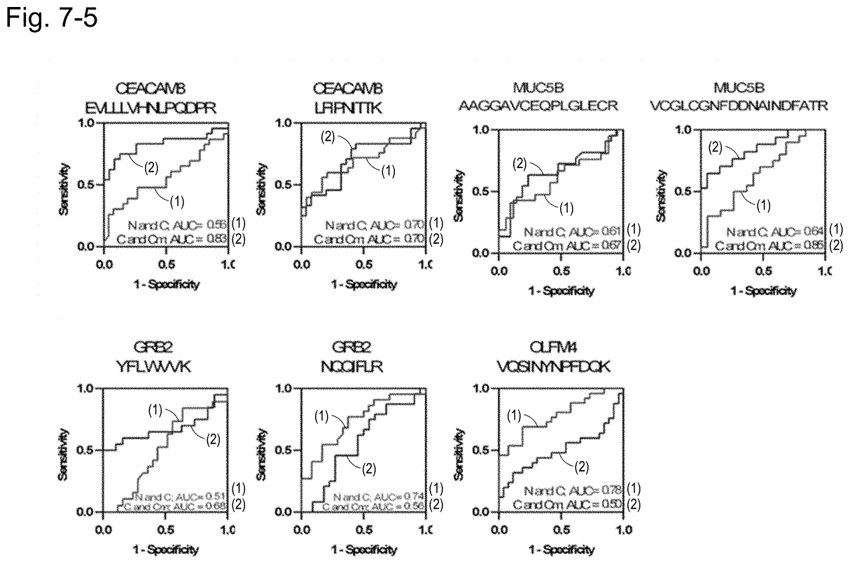

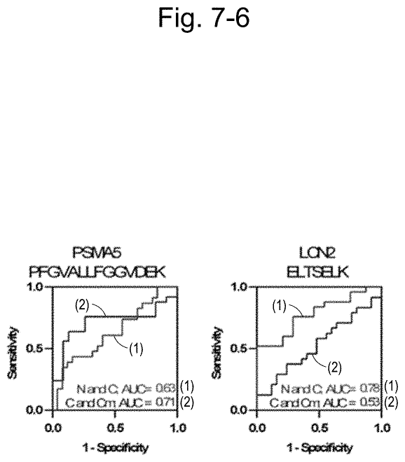

In order to evaluate the candidate peptides as biomarkers, 37 peptides were subjected to ROC (Receiver Operating Characteristic) analysis. Regarding individual candidate peptides, the distinguishing between N and C, and between C and Cm was evaluated. Four peptides (3 proteins) were found to be proteins having extremely high sensitivity (AUC>0.9, FIGS. 6-1a and b). Also, 22 peptides were found to have high sensitivity that was sufficient for distinguishing between N and C (0.7-0.9 AUC, FIGS. 7-1, 7-2, 7-3, 7-4, 7-5, and 7-6). On the other hand, 11 peptides had high sensitivity that was sufficient for distinguishing between C and Cm (FIGS. 6-2c and d).

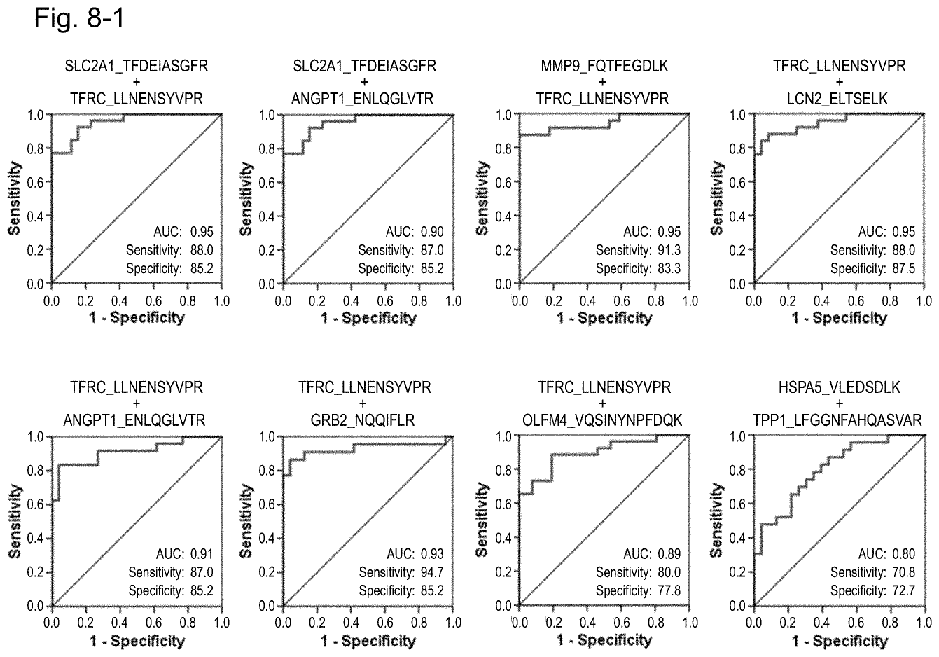

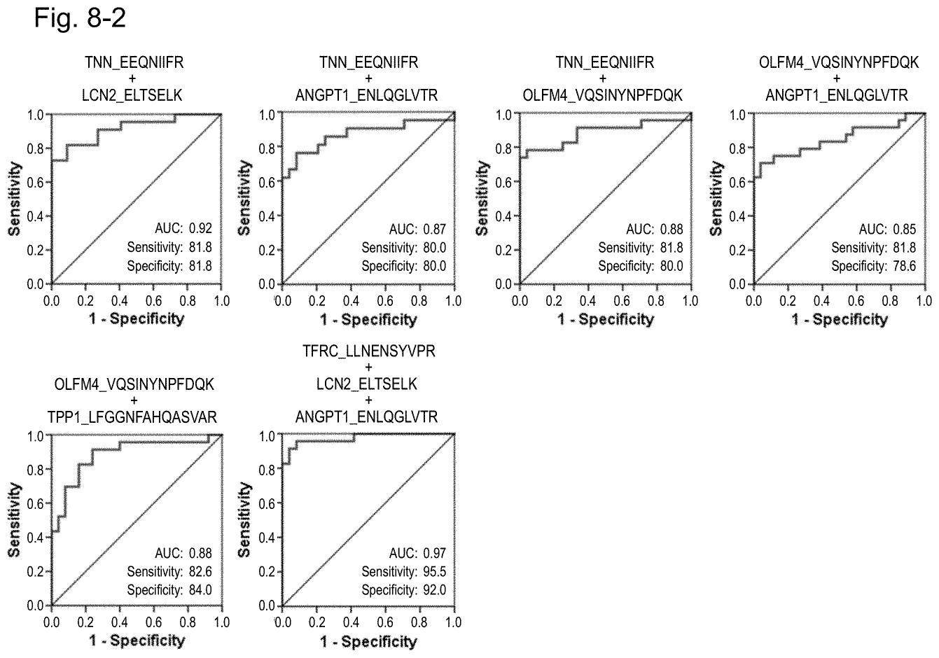

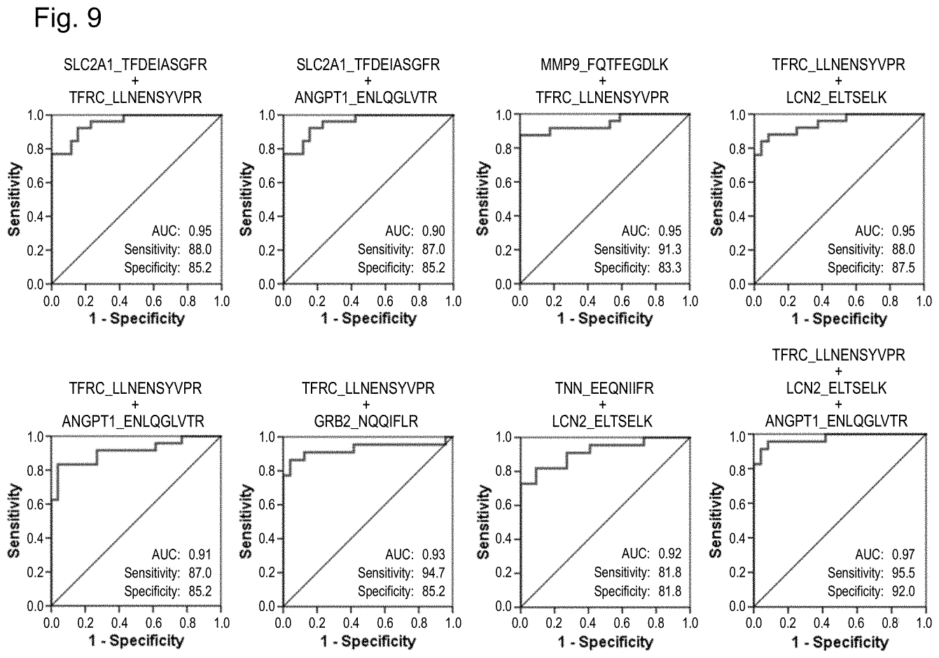

Subsequently, whether or not multiple markers obtained by combining candidate peptides with one another had high sensitivity was examined according to a logistic regression analysis. Significant combinations of peptides were evaluated using SPSS software. The results of the logistic analysis using two or more peptides were evaluated based on the AUC values of the ROC curves of the multiple markers. Regarding 14 combinations of candidate peptides, their AUC values were higher than the AUC value of a marker alone (FIGS. 8-1 and 8-2), and in particular, regarding 8 combinations, the AUC value was 0.9<(FIG. 9). The highest AUC value (0.97) was obtained by a combination of three peptides (Transferrin receptor protein 1, Neutrophil gelatinase-associated lipocalin, and Angiopoietin-1). These results suggest that a combination of multiple markers be effective for improving the accuracy of the diagnosis of colorectal cancer.

### Comparison Between Broadly Used Tumor Marker (Carcinoembryonic Antigen CEA) and Target Peptide in Terms of Sensitivity

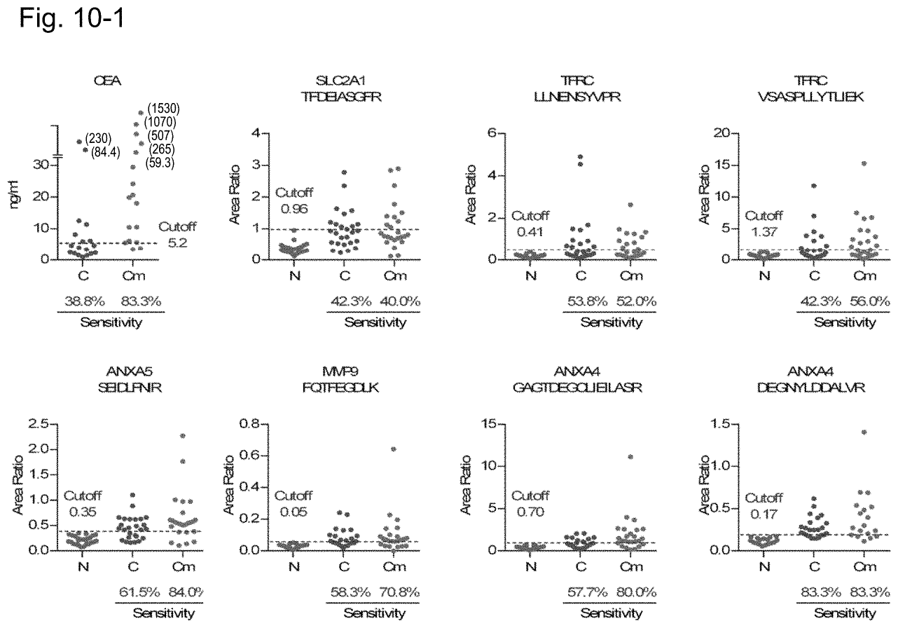

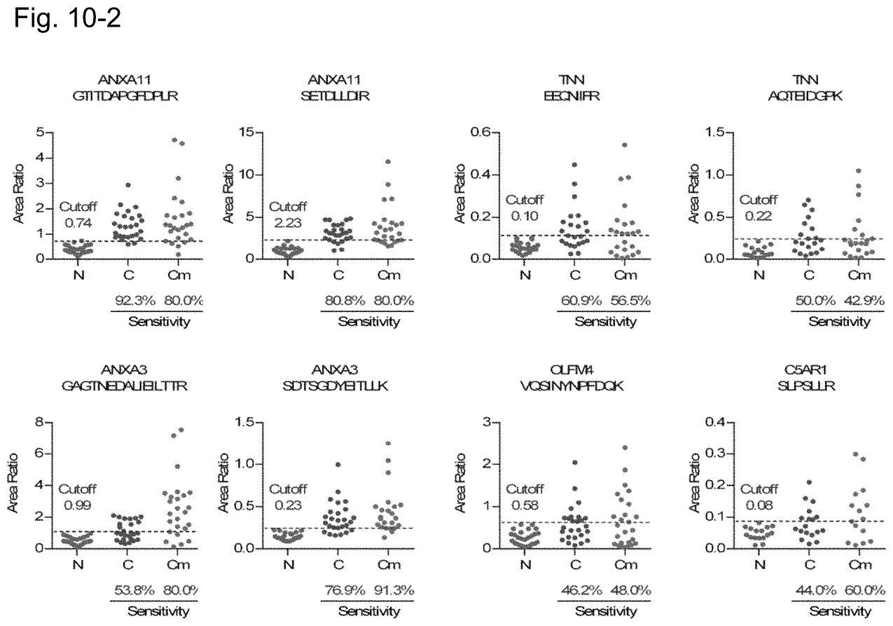

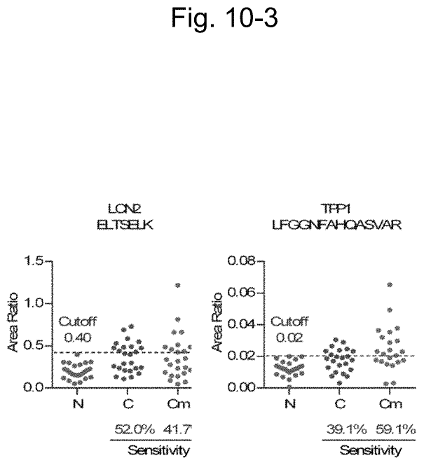

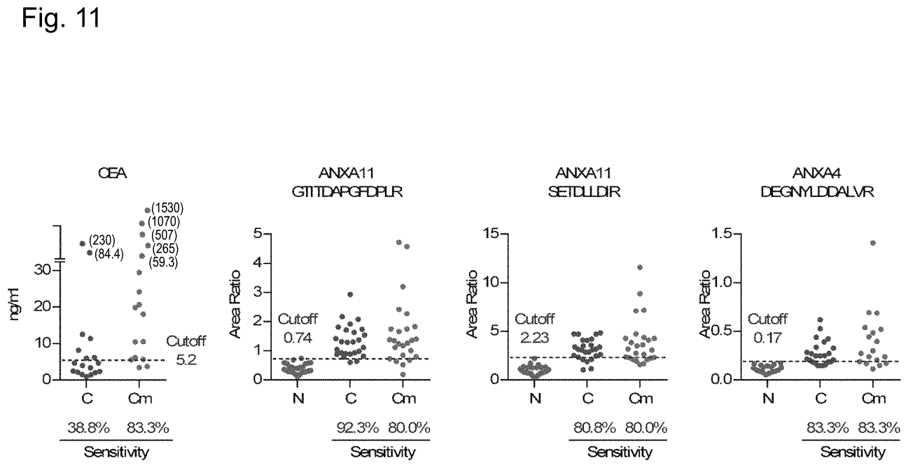

Today, CEA is one of most broadly used blood markers for colorectal cancer. However, the sensitivity of CEA is not sufficient for discovering early-stage patients, and the sensitivity of CEA to patients with stage 2 has been reported to be approximately 30%. Hence, newly established 37 biomarker candidates were compared with CEA. Since CEA specificity was almost 100%, the cutoff point of the SRM quantitative value of each target peptide was set to be a specificity of 100%. The sensitivity of CEA to specimen group C used in verification was 38.8%. On the other hand, among the 37 target peptides, 17 peptides (12 proteins; namely, Annexin A3, Annexin A4, Annexin A5, Annexin A11, Tenascin-N, Transferrin receptor protein 1, GLUT-1, Matrix metalloproteinase-9, Olfactomedin-4, CD88 antigen, Tripeptidyl-peptidase 1, and Neutrophil gelatinase-associated lipocalin) exhibited sensitivity that was significantly higher than the sensitivity of CEA (FIGS. 10-1, 10-2, and 10-3). In particular, the three peptides of Annexin A4 and A11 exhibited sensitivity of 80%<(FIG. 11).

### INDUSTRIAL APPLICABILITY

According to the present invention, colorectal cancer can be detected in an early stage.

### Sequence Listing Free Text

SEQ ID NOS: 23 to 59 Synthetic peptides

All publications, patents and patent applications cited in the present description are incorporated herein by reference in their entirety.

1. A colorectal cancer biomarker for detecting colorectal cancer, wherein the biomarker consists of at least one protein of the following 22 proteins with numbers 1 to 22, or at least one peptide of the partial peptides of the proteins with numbers 1 to 22:
1. Annexin A11 (ANXA 11) (SEQ ID NO: 1);
2. Annexin A3 (ANXA 3) (SEQ ID NO: 2);
3. Annexin A4 (ANXA 4) (SEQ ID NO: 3);
4. Tanascin-N(TNN) (SEQ ID NO: 4);
5. Transferrin receptor protein 1 (TFRC) (SEQ ID NO: 5);
6. Glucose transporter 1(GLUT-1) (SLC2A11) (SEQ ID NO: 6);
7. Complement component C9 (C9) (SEQ ID NO: 7);
8. CD88 antigen (C5AR1) (SEQ ID NO: 8);
9. 78 kDa glucose-regulated protein (HSPA5) (SEQ ID NO: 9);
10. α-1-acid glycoprotein (ORM1) (SEQ ID NO: 10);
11. Matrix metalloproteinase-9 (MMP9) (SEQ ID NO: 11);
12. Angiopoietin-1 (ANGPT1) (SEQ ID NO: 12);
13. CD67 antigen (CEACAM8) (SEQ ID NO: 13);
14. Mucin-5B (MUC5B) (SEQ ID NO: 14);
15. Adapter protein GRB2 (GRB2) (SEQ ID NO: 15);
16. Annexin A5 (Annexin A5) (ANXA 5) (SEQ ID NO: 16);
17. Olfactomedin-4 (OLFM4) (SEQ ID NO: 17);
18. Neutral amino acid transporter B(0) (SLC1A5) (SEQ ID NO: 18);
19. Tripeptidyl-peptidase 1 (TPP1) (SEQ ID NO: 19);
20. Heat shock-related 70 kDa protein 2 (HSPA2) (SEQ ID NO: 20);
21. Proteasome subunit α type-5 (PSMA5) (SEQ ID NO: 21); or
22. Neutrophil gelatinase-associated lipocalin (LCN2) (SEQ ID NO: 22).

2. The colorectal cancer biomarker for detecting colorectal cancer according to claim 1, which consists of a combination of two or more proteins of the 22 proteins with numbers 1 to 22 according to claim 1, or two or more peptides of the partial peptides of the proteins with numbers 1 to 22.

3. The colorectal cancer biomarker for detecting colorectal cancer according to claim 1, wherein the partial peptides of the proteins with numbers 1 to 22 according to claim 1 are peptides consisting of the amino acid sequences as set forth in SEQ ID NOS: 23 to 59.

4. The colorectal cancer biomarker for detecting colorectal cancer according to claim 1, which consists of at least one protein of the following 12 proteins, or at least one peptide of the partial peptides of the 12 proteins:
1. Annexin A11 (SEQ ID NO: 1);
2. Annexin A3 (SEQ ID NO: 2);
3. Annexin A4 (SEQ ID NO: 3);
4. Tanascin-N(SEQ ID NO: 4);
5. Transferrin receptor protein 1 (SEQ ID NO: 5);
6. Glucose transporter 1 (SEQ ID NO: 6);
8. CD88 antigen (SEQ ID NO: 8);
11. Matrix metalloproteinase-9 (SEQ ID NO: 11);
16. Annexin A5 (SEQ ID NO: 16);
17. Olfactomedin-4 (SEQ ID NO: 17);
19. Tripeptidyl-peptidase 1 (SEQ ID NO: 19); or
22. Neutrophil gelatinase-associated lipocalin (SEQ ID NO: 22).

5. The colorectal cancer biomarker for detecting colorectal cancer according to claim 4, which consists of a combination of two or more proteins of the 12 proteins according to claim 4, or two or more peptides of the partial peptides of the 12 proteins.

6. The colorectal cancer biomarker for detecting colorectal cancer according to claim 4, wherein the partial peptides of the 12 proteins according to claim 4 are peptides consisting of the amino acid sequences as set forth in SEQ ID NOS: 23 to 34, 37, 38, 43, 44, 53, 54, 56 and 59.

7. The colorectal cancer biomarker for detecting colorectal cancer according to claim 1, which consists of Annexin A4 or Annexin A11, or a partial peptide thereof.

8. The colorectal cancer biomarker for detecting colorectal cancer according to claim 7, which consists of a combination of Annexin A4 and Annexin A11, or partial peptides of Annexin A4 and partial peptides of Annexin A11.

9. The colorectal cancer biomarker for detecting colorectal cancer according to claim 7, wherein the partial peptides of Annexin A4 and Annexin A11 are peptides consisting of the amino acid sequences as set forth in SEQ ID NOS: 23, 24, 27 and 28.

10. A colorectal cancer biomarker for detecting colorectal cancer, which consists of a combination of the colorectal cancer biomarker according to claim 1 and CEA.

11. A method of detecting colorectal cancer, comprising measuring the colorectal cancer biomarker according to claim 1 in a biological sample.

12. A method of detecting colorectal cancer, comprising measuring the colorectal cancer biomarker according to claim 1 in a biological sample, and then determining that the subject is affected with colorectal cancer when the biomarker is present in a higher concentration in the biological sample than in a healthy subject.

13. The method of detecting colorectal cancer according to claim 11, wherein the biological sample is an extracellular vesicle (EV) in blood.

14. The method of detecting colorectal cancer according to claim 11, wherein the detection is carried out by an immunoassay or a mass spectrometry.

15. A kit of detecting colorectal cancer, comprising an antibody reacting against the colorectal cancer biomarker according to claim 1 in the biological sample.
